(19) United States
(12) Patent Application Publication
(10) Pub. No.: US 2025/0115881 A1
(43) Pub. Date: Apr. 10, 2025

(54) MODIFIED VERO CELLS AND METHODS OF USING THE SAME FOR VIRUS PRODUCTION

(71) Applicant: SANOFI PASTEUR, LYON (FR)

(72) Inventors: MARIE-ANGÉLIQUE ND¿YE, YANDÉ S¿NE, QUÉBEC (CA); AMINE ABDELKADER KAMEN, QUÉBEC (CA); JENNIFER REID, LYON (FR); TAO YUAN, LYON (FR)

(21) Appl. No.: 18/832,394

(22) PCT Filed: Jan. 25, 2023
(86) PCT No.: PCT/EP2023/051807

§ 371 Date: Jul. 23, 2024

(30) Foreign Application Priority Data
Jan. 27, 2022 (EP) 22305092.3

(51) Int. Cl. C12N 7/00; C12N 5/071; C12N 9/22; C12N 15/11; C12N 15/90

(52) U.S. Cl. CPC C12N 7/00; C12N 5/0686; C12N 9/22; C12N 15/11; C12N 15/907; C12N 2510/02; C12N 2760/18521; C12N 2760/18552; C12N 2760/20221; C12N 2760/20252

(57) ABSTRACT

Disclosed herein is an engineered cell line comprising a modification in an ISG15 gene, wherein the modification in the ISG15 gene results in an increase in total viral particle production and/or infectious viral particle production as compared to a control cell line that is identical to the engineered cell line except for the modification in the ISG15 gene. Also disclosed herein are methods of increasing viral particle production and methods of identifying a gene for deletion in a cell or cell line.

### FIELD OF THE DISCLOSURE

Disclosed herein are engineered cell lines for producing viral particles and methods of manufacturing the engineered cell lines. Also disclosed herein are methods of increasing viral particle production in a cell line and methods of identifying a gene targeted for deletion in a cell line to enhance production of viral particles.

### BACKGROUND OF THE DISCLOSURE

Viruses for use in vaccines, including live attenuated and inactivated viruses, are often manufactured using host cells from cultured cell lines, including plant, yeast, or animal cell lines, such as insect or mammalian cell lines. Examples of mammalian cells include, but are not limited to COS-7 cells, human embryonic kidney (HEK) cells, such as HEK293 cells; baby hamster kidney (BHK) cells; Chinese hamster ovary (CHO) cells; mouse sertoli cells; African green monkey kidney (Vero) cells; human cervical carcinoma cells (e.g., HeLa); canine kidney cells (e.g., MDCK), and the like.

These various cell culture-based platforms are used to produce viral vaccines as an alternative to egg-based vaccine production methods. The ability to increase the viral production rate may therefore enhance a cell line's potential for wider use.

Vero cells are a female African Green Monkey kidney-derived cell line that has been widely used for over 40 years in the production of viral vaccines, including, for example, vaccines against dengue fever, influenza, Japanese encephalitis, polio, rabies, rotavirus, smallpox, and Ebola (using a Vesicular Stomatitis Virus (VSV) recombinant virus (rVSV)). Furthermore, Vero cells have been identified as a cell line having a high susceptibility to coronaviruses, including, for example, MERS-COV, SARS-COV, and SARS-COV-2. Liu et al., A recombinant VSV-vectored MERS-COV vaccine induces neutralizing antibody and T cell responses in rhesus monkeys after single dose immunization, Antiviral Res. 2018, 150:30-38; Hoffmann et al., SARS-COV-2 Cell Entry Depends on ACE2 and TMPRSS2 and Is Blocked by a Clinically Proven Protease Inhibitor, Cell 2020, 181 (2): 271-280. Thus, successfully engineering cell lines, including Vero cell lines, to increase viral production of viruses may greatly impact global health.

Thanks to the recent advances in gene editing, it is possible to edit the genome of cell lines and notably those used for viral infection studies and vaccine production for vaccine bioprocessing intensification purposes using the available genomic information. Previous attempts were made to increase viral production rates in Vero cells through gene editing using a genome-wide RNA interference screen dataset to choose gene targets, but no significant increase was observed after repeating the experiments. Van der Sanden, S. M. et al., Engineering Enhanced Vaccine Cell Lines To Eradicate Vaccine-Preventable Diseases: the Polio End Game, J. Virol. 2016, 90 (4): 1694-704; Hoeksema F. et al., Enhancing viral vaccine production using engineered knockout vero cell lines—A second look, Vaccine 2018, 36 (16): 2093-2103. Among the possible reasons cited by the authors to explain such results were the use of a genome other than the Vero cell genome and the fact that the phenotypes induced by transcriptional suppression (RNAi-based knockdown) and genetic deletion (Clustered Regularly Interspaced Short Palindromic Repeats (CRISPR) knockout) may differ in such a way that the former might increase virus production while the latter does not.

Thus, disclosed herein are methods to identify valuable target genes for editing that do not rely on genome-wide screens, as well as engineered cell lines wherein the desired gene target or targets have been modified, allowing for enhanced production of viral particles.

### SUMMARY OF THE DISCLOSURE

The present disclosure provides an engineered cell line comprising a modification in one or more genes that results in an increase in total viral particle production and/or infectious viral particle production as compared to a control cell line that is identical to the engineered cell line except for the modification in the one or more genes.

In certain embodiments, disclosed herein is an engineered cell line comprising a modification in an ISG15 gene that results in an increase in total viral particle production and/or infectious viral particle production as compared to a control cell line that is identical to the engineered cell line except for the modification in the ISG15 gene. In certain embodiments, the engineered cell line comprises a modification in one or more genes selected from APOA1, CCL2, CCL5, CYP19A1, CXCL8, ELF3, FOS, HERC3, HERC5, IFIT1, IFIT2, IFIT3, IRF7, ISG15, KRT15, KRT19, MX1, NGFR, PTGS2, PTPN6, RET, ROS1, SFRP1, SOX2, SPP1, TNF, TNFRSF4, TRAF1, and VAV3.

In one aspect of the disclosure, the increase in viral particle production is at least about 20%, such as at least about 40%, at least about 50%, at least about 60%, at least about 70%, at least about 80%, at least about 90%, at least about 100%, at least about 150%, at least about 200%, at least about 250%, or at least about 300% relative to the control cell line. In certain aspects, there is an increase in the ratio of infectious viral particle production to total viral particle production of at least about 3%, such as at least about 20%, at least about 30%, at least about 40%, at least about 50%, or at least about 60%, relative to the control cell line.

In certain embodiments, the modification in the one or more genes results in deletion of the one or more genes from the engineered cell line, and in certain embodiments, the engineered cell line comprises decreased expression of the one or more genes as compared to the control cell line. For instance, in certain embodiments, the modification in the ISG15 gene results in deletion of the ISG15 gene from the engineered cell line, and in certain embodiments, the engineered cell line comprises decreased expression of the ISG15 gene as compared to a control cell line.

In certain aspects, the engineered cell line is from a monkey cell or a mouse cell. In certain aspects, the engineered cell line is a Vero cell.

In certain aspects, the one or more genes is modified or deleted from the engineered cell line using a CRISPR-associated (Cas) system. In one aspect of the disclosure, the ISG15 gene is modified or deleted from the engineered cell line using a CRISPR-Cas system.

In certain embodiments of all aspects of the disclosure, the virus is selected from influenza virus, dengue virus, yellow fever virus, respiratory syncytial virus (RSV), herpes simplex virus, human immunodeficiency virus (HIV), hepatitis virus, coronavirus, or a virus from the Rhabdoviridae family, such as rabies virus or vesicular stomatitis virus (VSV). In certain aspects, the virus is an influenza virus, such as an influenza A virus or an influenza B virus.

Also disclosed herein are methods of increasing viral particle production comprising infecting an engineered cell line with a virus, incubating the engineered cell line under conditions suitable for production of the virus by the engineered cell line, and harvesting the virus produced by the engineered cell line, wherein the engineered cell line comprises a modification in one or more genes, such as an ISG15 gene, wherein the modification in the one or more genes, such as the ISG15 gene, results in an increase in total viral particle production and/or infectious viral particle production as compared to a control cell line that is identical to the engineered cell line except for the modification in the one or more genes, such as the ISG15 gene.

In certain embodiments of the methods disclosed herein, the engineered cell line increases viral particle production by at least about 20%, such as at least about 40%, at least about 50%, at least about 60%, at least about 70%, at least about 80%, at least about 90%, at least about 100%, at least about 150%, at least about 200%, at least about 250%, or at least about 300% relative to the control cell line. In certain embodiments, the modification in the one or more genes, such as the ISG15 gene, results in deletion of the one or more genes from the engineered cell line, and in certain embodiments, the engineered cell line comprises decreased expression of the one or more genes, such as the ISG15 gene, from the engineered cell line. In certain embodiments of the methods disclosed herein, the engineered cell line is a Vero cell line, and in certain embodiments, the virus is selected from influenza virus, dengue virus, yellow fever virus, RSV, herpes simplex virus, HIV, hepatitis virus, coronavirus, or a virus from the Rhabdoviridae family, such as rabies virus or VSV.

Also disclosed herein is a method of identifying a gene for deletion in a cell or a cell line, the method comprising (1) infecting the cell or cell line with a virus; (2) detecting expression levels of multiple genes in the infected cell or cell line and comparing the expression levels to expression levels of the multiple genes in a control cell or cell line that is not infected with the virus; (3) identifying the gene targets that are differentially expressed in the infected cell or cell line; (4) analyzing the differentially expressed gene targets to identify one or more gene targets involved in multiple protein-protein networks, wherein the multiple protein-protein networks comprise at least two of defense response, response to virus, viral genome replication, response to cytokine, response to type I interferon, regulation of viral genome replication, defense response to virus, cell death, viral life cycle, negative regulation of viral genome replication, and cellular response to cytokine stimulus; and (5) selecting at least one differentially expressed gene target for deletion in the cell or cell line, wherein deletion of the at least one differentially expressed gene target increases viral particle production of the virus. In certain embodiments, the method further comprises (6) deleting the at least one differentially expressed gene target from the cell or cell line using a gene editing technique, such as a CRISPR-Cas system.

In certain embodiments of the methods of identifying a gene for deletion in a cell or a cell line, the cell or cell line is a Vero cell, a Madin-Darby Canine (MDCK) cell, or a Human Embryonic Kidney (HEK) cell. In certain embodiments, the cell or cell line is a Vero cell. In certain embodiments of the methods, the virus is selected from influenza virus, dengue virus, yellow fever virus, RSV, herpes simplex virus, HIV, hepatitis virus, coronavirus, or a virus from the Rhabdoviridae family, such as rabies virus or VSV. In certain embodiments, the virus is an influenza virus, such as an influenza A virus or an influenza B virus.

### BRIEF DESCRIPTION OF THE DRAWINGS

FIG. 1 is a graph showing normalized expression scores from the gene set enrichment analysis (GSEA) for influenza-infected Vero cells 24 hours post-infection, as described in Example 2.

FIG. 2 is a graph showing normalized expression scores from the GSEA for rVSV-GFP infected Vero cells 6 hours post-infection, as described in Example 2.

FIG. 3 is a sequence alignment comparing human ISG15 (hISG15) (SEQ ID NO. 15), mouse ISG15 (mISG15) (SEQ ID NO. 16), Vero cell ISG15 (vISG15) (SEQ ID NO. 14), and canine ISG15 (caISG15) (SEQ ID NO. 17), as described in Example 3. The amino acid residues known to interact with influenza NS1 protein, coronavirus PLP, and nairovirus OTUs are shown below the sequence alignment.

FIG. 4 is a schematic illustrating the location of PCR primers sgRNA A and sgRNA B, as described in Example 4, for the detection of non-deletion and deletion bands, respectively.

FIG. 5 is an image of a PCR screen showing non-deletion and deletion bands for parental Vero cells (left) and ISG15−/− Vero cells (right), as described in Example 4.

FIG. 6 is a Western Blot of parental Vero cells and ISG15−/− Vero cell, showing the detection of ISG−/− Vero cells as indicated by the absence of a band at about 17 kDa and as described in Example 4.

FIG. 7 is a graph showing viral genome/mL and TCID50/mL for parental Vero cells infected with influenza virus A (IVA), ISG15−/− Vero cells infected with IVA, parental Vero cells infected with rVSV-GFP, and ISG15−/− Vero cells infected with rVSV-GFP, as described in Example 5.

FIG. 8 is a graph showing a growth rate analysis for an ISG15−/− cell line and control cell line in serum-free media, wherein dt is doubling time and the lines are drawn as comparison of fits for nonlinear fit, as described in Example 6.

FIG. 9A is a graph showing the number of viable cells over time post-infection for ISG15−/− (p5+4), ISG15−/− (p17), and control cell lines in serum free media in bioreactors, as described in Example 7.

FIG. 9B is a graph showing the percent of cell viability over time post-infection for ISG15−/− (p5+4), ISG15−/− (p17), and control cell lines in serum free media in bioreactors, as described in Example 7.

FIG. 10A are plots showing the log titre (PFU/mL) over time post-infection (left) and titre (PFU/mL) over time post-infection (right) for ISG15−/− (p5+4), ISG15−/− (p17), and control cell lines in serum free media in bioreactors, as described in Example 8.

FIG. 10B is a plot showing the log titre per 10⁶ cells 3 days post-infection for ISG15−/− (p5+4), ISG15−/− (p17), and control cell lines in serum free media in bioreactors, as described in Example 8.

### DETAILED DESCRIPTION OF THE DISCLOSURE

Disclosed herein is the use of a cell line, such as the Vero cell line, to identify various factors at play during viral infection and reproduction in host cells in order to enhance production of viral particles, for example for use in manufacturing vaccine compositions. In certain embodiments, a candidate antiviral gene for gene editing may be selected using any combination of methods, including differential gene expression analysis, Gene Set Enrichment Analysis (GSEA), and/or analysis of protein-protein interactions (e.g., Network Topology Analysis), for example. After selection of a target gene or target genes for genomic editing, sequences may be analyzed for gene editing. For example, sequences may be isolated for identification of CRISPR guide RNA (gRNA) design and off-target predictions in order to further reduce potential gRNA candidates to those with the highest knockout efficiency score. Thereafter, an engineered cell line may be designed wherein the target gene or genes has been modified to allow for enhanced production of viral particles in an infected engineered cell line.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Approximately: As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

CRISPR-Cas system: As used herein, a “CRISPR-associated (Cas) system” or “CRISPR-Cas system” refers to transcripts and other elements involved in the expression of or directing the activity of Cas genes, including sequences encoding a Cas gene, such as Cas9. In general, a CRISPR system contains elements that promote the formation of a CRISPR complex at the site of a target sequence (e.g., a protospacer). A guide sequence is designed to have complementarity to the target sequence, where hybridization between the target sequence and the guide sequence promotes the formation of the CRISPR complex. A target sequence may comprise any polynucleotide, such as DNA or RNA polynucleotides.

sgRNA: As used herein, the terms “guide RNA”, “single guide RNA,” or “sgRNA” refer to any polynucleotide sequence, such as DNA or RNA polynucleotides, having sufficient complementarity with a target sequence to hybridize with the target sequence and direct sequence-specific binding of a CRISPR complex to the target sequence. In some embodiments, the degree of complementarity, when optimally aligned using a suitable alignment algorithm, is at least about 50%, such as at least about 60%, at least about 70%, at least about 80%, at least about 90%, at least about 95%, at least about 99%, or about 100%. Optimal alignment may be determined with the use of any suitable algorithm for aligning sequences, including, for example, the Smith-Waterman algorithm, the Needleman-Wunsch algorithm, the Burrows Wheeler Aligner, ClustalW, Clustal X, BLAST, Novoalign, ELAND (Illumina, San Diego, Calif.), SOAP (available at soap.genomics.org.cn), and Maq (available at maq.sourceforge.net).

Gene: As used herein, the terms “gene” or “genes” refers to a nucleic acid (e.g., DNA or RNA) sequence comprising coding sequences for the production of a polypeptide or precursor (e.g., a protein). The polypeptide can be encoded by a full length coding sequence or by any portion of the coding sequence so long as the desired activity or functional properties of the full-length or fragment are retained. The term “gene” encompasses both cDNA and genomic forms of a gene.

Gene expression: The term “gene expression” refers to the expression level of a gene in a sample. As is understood in the art, the expression level of a gene can be analyzed by measuring the expression of a nucleic acid (e.g., mRNA or cDNA) or a polypeptide that is encoded by the nucleic acid. Gene expression may be up-regulated, indicating that the expression level of the gene in a sample is increased as compared to a normalized gene expression, or down-regulated, indicating that the expression level of the gene in a sample is decreased as compared to a normalized gene expression.

Normalized gene expression: The term “normalized gene expression” refers to an average gene expression level for a given gene in a sample or a pool of samples that are free of the disease or virus.

Pharmaceutically acceptable carrier: The term “pharmaceutically acceptable carrier” or “pharmaceutically acceptable excipient” means solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. In certain embodiments, the pharmaceutically acceptable carrier or excipient is not naturally occurring.

Polypeptide: The terms “polypeptide,” “peptide,” and “protein” are used interchangeably herein to refer to polymers of amino acids.

Primer: The term “primer” means a polynucleotide capable of binding to a region of a target nucleic acid, or its complement, and promoting nucleic acid amplification of the target nucleic acid. Generally, a primer will have a free 3′ end that can be extended by a nucleic acid polymerase. Primers also generally include a base sequence capable of hybridizing via complementary base interactions either directly with at least one strand of the target nucleic acid or with a strand that is complementary to the target sequence. A primer may comprise target-specific sequences and optionally other sequences that are non-complementary to the target sequence. These non-complementary sequences may comprise, for example, a promoter sequence or a restriction endonuclease recognition site.

Subject: The term “subject” refers to any animal, such as a mammal, including humans, non-human primates, rodents, and the like which is to be the recipient of a particular treatment. In some embodiments, “subject” refers to humans. In some embodiments, “subject” refers to non-human animals.

Vaccine: As used herein, the term “vaccine” refers to a composition administered to trigger or stimulate an immune response against a particular disease, such as an influenza infection. The term vaccine comprises preventative vaccines and therapeutic vaccines. Preventative vaccines are designed to prevent a subject from acquiring a particular disease, such as influenza infection, or to only have a mild case of the disease. Therapeutic vaccines are intended to improve immune response to or alleviate symptoms of specific diseases.

Viral infection: As used herein, the term “viral infection” describes a diseased state in which a virus invades healthy cells, uses the cell's reproductive machinery to multiply or replicate and ultimately lyse the cell, resulting in cell death, release of viral particles, and the infection of other cells by the newly produced progeny viruses. Latent infection by certain viruses, e.g., HIV-1, is also a possible result of viral infection.

Viral particle: As used herein, a “viral particle” is a virion that replicates inside of a living cell. Viral particles contain genetic material (i.e., DNA or RNA), a protein coat, or capsid, that surrounds the genetic material, and optionally a lipid envelop.

Infectious viral particle: As used herein, “infectious viral particles” are viral particles that have a cytopathic effect on a host cell. Infectious viral particles may be quantified by any method known in the art, including, for example, by using a Median Tissue Culture Infectious Dose (TCID50) assay and/or using a plaque assay, as described herein.

Disclosed herein are various genes. The following Table 1 lists gene names, species, NCBI References Sequence numbers, and descriptions for genes discussed herein. This listing does not constitute a complete listing of genes that may be contemplated and are considered within the scope of the present disclosure.

TABLE 1
NCBI Gene Sequences

| Gene | | | | |
|---|---|---|---|---|
| Name | Species | NCBI Ref. Seq. | Description | Sequence |
| APOA1 | Chlorocebus | XP_008019172.2 | Apolipoprotein A1 | SEQ ID NO: 1 |
| | sabaeus | | | |
| CCL2 | Chlorocebus | XP_008009200.2 | C—C motif chemokine 2 | SEQ ID NO: 2 |
| | sabaeus | | | |
| CCL5 | Chlorocebus | XP_008009272.2 | C—C motif chemokine 2 | SEQ ID NO: 3 |
| | sabaeus | | | |
| CYP19A1 | Chlorocebus | XP_008014801 | Cytochrome P450 | SEQ ID NO: 4 |
| | sabaeus | | family 19 subfamily A | |
| | | | member 1 | |
| CXCL8 | Chlorocebus | NP_001279010 | C—X—C motif | SEQ ID NO: 5 |
| | sabaeus | | chemokine ligand 8 | |
| ELF3 | Chlorocebus | XP_007987143.2 | ETS-related | SEQ ID NO: 6 |
| | sabaeus | | transcription factor | |
| | | | Elf-3 | |
| FOS | Chlorocebus | XP_007985512.1 | Fos proto-oncogene, | SEQ ID NO: 7 |
| | sabaeus | | AP-1 transcription | |
| | | | factor | |
| HERC3 | Chlorocebus | XP_007997428 | HECT and RLD | SEQ ID NO: 8 |
| | sabaeus | | domain containing E3 | |
| | Chlorocebus | | ubiquitin protein | |
| | sabaeus | | ligase 3 | |
| HERC5 | Chlorocebus | XP_007997417 | HECT and RLD | SEQ ID NO: 9 |
| | sabaeus | | domain containing E3 | |
| | | | ubiquitin protein | |
| | | | ligase 5 | |
| IFIT1 | Chlorocebus | XP_007961691.1 | Interferon induced | SEQ ID NO: 10 |
| | sabaeus | | protein with | |
| | | | tetratricopeptide | |
| | | | repeats 1 | |
| IFIT2 | Chlorocebus | XP_0079687.1 | Interferon induced | SEQ ID NO: 11 |
| | sabaeus | | protein with | |
| | | | tetratricopeptide | |
| | | | repeats 2 | |
| IFIT3 | Chlorocebus | XP_037865171.1 | Interferon induced | SEQ ID NO: 12 |
| | sabaeus | | protein with | |
| | | | tetratricopeptide | |
| | | | repeats 3 | |
| IRF7 | Chlorocebus | XP_007982588.1 | Interferon regulatory | SEQ ID NO: 13 |
| | sabaeus | | factor 7 | |
| ISG15 | Chlorocebus | XP_007979280.1 | ISG15 ubiquitin like | SEQ ID NO: 14 |
| | sabaeus | | modifier | |
| ISG15 | Homo | NP_005092.1 | ISG15 ubiquitin like | SEQ ID NO: 15 |
| | sapiens | | modifier | |
| ISG15 | Mus | NP_056598.2 | ISG15 ubiquitin like | SEQ ID NO: 16 |
| | musculus | | modifier | |
| ISG15 | Canis lupus | XP_003639101.1 | ISG15 ubiquitin like | SEQ ID NO: 17 |
| | familiaris | | modifier | |
| KRT15 | Chlorocebus | XP_008010906.1 | Keratin 15 | SEQ ID NO: 18 |
| | sabaeus | | | |
| KRT19 | Chlorocebus | XP_008010904.1 | Keratin 19 | SEQ ID NO: 19 |
| | sabaeus | | | |
| MX1 | Chlorocebus | XP_007966906.2 | MX dynamin like | SEQ ID NO: 20 |
| | sabaeus | | GTPase 1 | |
| NGFR | Chlorocebus | XP_008011253.1 | Nerve growth factor | SEQ ID NO: 21 |
| | sabaeus | | receptor | |
| PTGS2 | Chlorocebus | XP_007987337.1 | Prostaglandin- | SEQ ID NO: 22 |
| | sabaeus | | endoperoxide | |
| | | | synthase 2 | |
| PTPN6 | Chlorocebus | XP_037844268.1 | Protein tyrosine | SEQ ID NO: 23 |
| | sabaeus | | phosphatase non- | |
| | | | receptor type 6 | |
| RET | Chlorocebus | XP_007960625.1 | Ret proto-oncogene | SEQ ID NO: 24 |
| | sabaeus | | | |
| ROS1 | Chlorocebus | XP_037856910.1 | ROS proto-oncogene 1, | SEQ ID NO: 25 |
| | sabaeus | | receptor tyrosine | |
| | | | kinase | |
| SFRP1 | Chlorocebus | XP_007960468.1 | Secreted frizzled | SEQ ID NO: 26 |
| | sabaeus | | related protein 1 | |
| SOX2 | Chlorocebus | XP_007970196.1 | SRY-box transcription | SEQ ID NO: 27 |
| | sabaeus | | factor 2 | |
| SPP1 | Chlorocebus | XP_007997394.1 | Sabaeus secreted | SEQ ID NO: 28 |
| | sabaeus | | phosphoprotein 1 | |
| TNF | Chlorocebus | XP_037861775.1 | Tumor necrosis factor | SEQ ID NO: 29 |
| | sabaeus | | | |
| TNFRSF4 | Chlorocebus | XP_007979248.1 | TNF receptor | SEQ ID NO: 30 |
| | sabaeus | | superfamily member 4 | |
| TRAF1 | Chlorocebus | XP_007966416.1 | TNF receptor | SEQ ID NO: 31 |
| | sabaeus | | associated factor 1 | |
| VAV3 | Chlorocebus | XP_007975962.2 | Vav guanine | SEQ ID NO: 32 |
| | sabaeus | | nucleotide exchange | |
| | | | factor 3 | |

Presented herein is an analysis of virus-host interactions at play during viral infection, as host cells attempt to minimize the impact of viral infection, and viruses attempt to evade host cell immune responses. Through the use of an interdisciplinary approach combining functional genomics and cell biology, disclosed herein is a new strategy for a more efficient, targeted gene editing, in order to enhance viral particle production in host cells and thereby open up new possibilities for host cells, including Vero cells, for pandemic-ready, high throughout vaccine production platforms.

The de novo assembled and annotated Vero genome was recently published. Sène, M.-A. et al., Haplotype-resolved de novo assembly of the Vero cell line genome, NPJ Vaccine 2021, 6 (1): 106. Based on this information and together with the use of functional genomics, better control and oversight on the effects of gene editing of Vero cells are possible, which allows for a deeper understanding of the gene target candidates before selection, as well as a better understanding of the mechanisms at play during infection. The deletion of a whole genomic region, such as the coding region (the coding DNA sequence, or CDS region), may increase the probability of getting a biallelic deletion as compared to gene knockdown using a single guide RNA-based cut. This increases the probability that the deletion will lead to the desired loss of function of the targeted gene product or products and also simplifies validation of gene knockout, ensuring a rapid and high throughput gene editing protocol.

As disclosed herein, deletion of ISG15 in Vero cells led to an overall increase in total viral particle production as well as an increase of infectious viral particle production and an increase in the ratio of infectious viral particles to total viral particles.

ISG15 is a 17 kDa antiviral protein (15 kDa after maturation by N-terminal Met excision and removal of C-terminal peptide) that protects the host cell from viral infection via the inhibition of viral replication in a conjugation-dependent manner. Pattyn E. et al., HyperISGylation of Old World monkey ISG15 in human cells, PLOS One 2008; 3 (6): e2427. Although ISG15 has been implicated in antiviral responses to various viruses, including SARS, influenza, HIV, and hepatitis, there is a functional diversity of ISG15 across species, as ISG15-deficient human patients show no increased susceptibility to viral infection, as compared to ISG15-deficient mice, which are more susceptible to viral infections. Perng, Y. C. et al., ISG15 in antiviral immunity and beyond, Nat Rev Microbiol. 2018, 16 (7): 423-439. It is therefore desirable to consider diversity across cell species to ensure that gene editing will lead to the desired phenotypic modifications with regards to viral infection. As such, protein sequences may be compared between species of interest (including from species of cells used in vaccine production, such as Vero, HEK293 and MDCK).

Accordingly, disclosed herein are engineered cell lines comprising a modification in one or more genes, such as a modification in an ISG15 gene of a Vero cell line, wherein the modification results in an increase in total viral particle production and/or infectious viral particle production as compared to a control cell line. Also disclosed are methods of making the engineered cell line and methods of using the engineered cell line to increase viral particle production. Additionally, disclosed herein are methods of identifying a target gene for modification (such as deletion) in a cell or cell line.

### Engineered Cell Lines

In certain embodiments, disclosed herein is an engineered cell line comprising a modification in one or more genes, wherein the modification in the one or more genes results in an increase in total viral particle production and/or infectious viral particle production as compared to a control cell line that is identical to the engineered cell line except for the modification or modifications. As used herein, a control cell line, which is identical to the engineered cell line except for the modification or modifications, may be a parental cell line to the engineered cell line (i.e., originating from a same cell culture). Accordingly, in certain embodiments, the control cell line is a parental cell line. In certain embodiments, the engineered cell line is a Vero cell line, and in certain embodiments, the modification is a modification of an ISG15 gene, such as deletion of the ISG15 gene.

In some embodiments, the engineered cell line comprises a modification of one or more of the following genes: APOA1, CCL2, CCL5, CYP19A1, CXCL8, ELF3, FOS, HERC3, HERC5, IFIT1, IFIT2, IFIT3, IRF7, ISG15, KRT15, KRT19, MX1, NGFR, PTGS2, PTPN6, RET, ROS1, SFRP1, SOX2, SPP1, TNF, TNFRSF4, TRAF1, and VAV3 as compared to a control cell line. In some embodiments, the engineered cell line comprises a modification of one or more of the following genes: CCL2, CCL5, CXCL8, HERC5, IFIT1, IFIT2, IFIT3, and ISG15 as compared to a control cell line. In certain embodiments, the engineered cell line comprises a modification of the ISG15 gene.

As used herein, a modification refers to any non-natural permutation of the genome of the cell line that affects a gene or genes' expression in the cell line. For example, a modification to a gene may increase expression of that gene, or, in certain embodiments, a modification may decrease expression of that gene. In certain embodiments, a modification may comprise deletion of a part of the gene or the entire gene from the genome of the cell line, including deletion of the CDS region.

Decreased expression refers to decreased transcription of a coding region of a gene, a decrease in translation of the mRNA encoded by the coding region, or a decrease in activity of the resultant protein encoded by the coding region. Increased expression refers to increased transcription of a coding region of a gene, an increase in translation of the mRNA encoded by the coding region, or an increase in activity of the resultant protein encoded by the coding region.

For instance, in certain embodiments, the modification comprises deletion of one or more of the following genes from the engineered cell line: APOA1, CCL2, CCL5, CYP19A1, CXCL8, ELF3, FOS, HERC3, HERC5, IFIT1, IFIT2, IFIT3, IRF7, ISG15, KRT15, KRT19, MX1, NGFR, PTGS2, PTPN6, RET, ROS1, SFRP1, SOX2, SPP1, TNF, TNFRSF4, TRAF1, and VAV3, and in certain embodiments, the modification comprises deletion of one or more of the following genes from the engineered cell line: CCL2, CCL5, CXCL8, HERC5, IFIT1, IFIT2, IFIT3, and ISG15. In certain embodiments, the modification comprises deletion of the ISG15 gene from the engineered cell line.

As is known in the art, a cell line is a clonal cell culture developed from a single cell, wherein the culture of cells continues to divide for an extended period time without undergoing senescence. A control cell line differs from an engineered cell line in that the control cell line is derived from a same or similar clonal cell culture and is therefore genetically similar to the engineered cell line, but the control cell line has not been engineered to modify expression of the one or more target genes. In certain embodiments, the engineered cell line and the control cell line are selected from primate cells, such as a monkey cell or a human cell, a mouse cell, or a canine cell. For instance, in certain embodiments, the engineered cell line and the control cell line may be a Vero cell, a Madin-Darby Canine (MDCK), or a Human Embryonic Kidney (HEK) cell. In certain embodiments, the engineered cell line and the control cell line are Vero cells.

Engineered cell lines may be engineered to modify expression of the one or more genes in any manner known in the art. In certain embodiments, the engineered cell line is modified by editing the cellular genome, such as through the use of a CRISPR technology.

In certain embodiments, a CRISPR system comprises a Cas9 endonuclease and single guide RNA (“sgRNA”) to create an engineered cell line having one or more genes that are knocked-out or deleted. CRISPR stands for Clustered Regularly Interspaced Short Palindromic Repeats and is a system for genome engineering that may be used to knock out specific genes. sgRNAs are short guide RNAs that comprise a Cas9 endonuclease binder of approximately 20 nucleotides that may be used as the target sequence. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. “Target sequence” refers to a sequence to which a guide sequence is designed to have complementarity, where hybridization between the target sequence and the guide sequence promotes the formation of a CRISPR complex. A target sequence may comprise any polynucleotide, such as DNA or RNA polynucleotides. This target sequence can be altered to suppress or activate specific genes in order to create a customized knockout cell line. The Cas9/sgRNA complex acts by binding to the cellular DNA, cleaving it at the designated target spot, and then repairing the double-stranded break after cleavage. Shalem et al., Genome-Scale CRISPR-Cas9 Knockout Screening in Human Cells, SCIENCE, 343; 6166, 84-87 (2014). In this way, a specific gene knockout may be created.

In some embodiments, the expression of any of the selected target gene or genes is modified using a method comprising introducing into the cell a CRISPR/Cas endonuclease (Cas) 9 system with a CRISPR/Cas guide RNA, wherein the guide RNA targets the gene or a fragment thereof. Accordingly, in one aspect, disclosed here are methods of genetically engineering a cell line, such as a Vero cell line, comprising obtaining sgRNA specific for a target DNA sequence in the cell; and b) transducing (for example, introducing via electroporation) into a target cell, a CRISPR/Cas endonuclease (Cas9) complexed with a corresponding CRISPR/Cas guide RNA that hybridizes to the target sequence within the genomic DNA of the cell.

“Guide RNA”, “single guide RNA” and “synthetic guide RNA” are used interchangeably and refer to the polynucleotide sequence comprising the guide sequence, the tracr sequence and the tracr mate sequence. The term “guide sequence” refers to the about 20 bp sequence within the guide RNA that specifies the target site and may be used interchangeably with the terms “guide” or “spacer”. In some embodiments, the gRNA comprises a sequence of ACCAGCATTCGAGCAAGATCAAGG (SEQ ID NO: 33), and in some embodiments, the gRNA comprises a sequence of GGAAACCGAAACTTGGCCACCGG (SEQ ID NO: 34). In some embodiments, the CRISPR/Cas system comprises a first guide sequence of ACCAGCATTCGAGCAAGATCAAGG (SEQ ID NO: 33) and a second guide sequence of GGAAACCGAAACTTGGCCACCGG (SEQ ID NO: 34).

In certain embodiments disclosed herein, the one or more genes are deleted from the engineered cell line using a CRISPR-Cas system. For example, in certain embodiments, one or more of the following genes are deleted from the engineered cell line using a CRISPR-Cas system: APOA1, CCL2, CCL5, CYP19A1, CXCL8, ELF3, FOS, HERC3, HERC5, IFIT1, IFIT2, IFIT3, IRF7, ISG15, KRT15, KRT19, MX1, NGFR, PTGS2, PTPN6, RET, ROS1, SFRP1, SOX2, SPP1, TNF, TNFRSF4, TRAF1, and VAV3, and in certain embodiments, one or more of the following genes are deleted from the engineered cell line using a CRISPR-Cas system: CCL2, CCL5, CXCL8, HERC5, IFIT1, IFIT2, IFIT3, and ISG15. In certain embodiments, the ISG15 gene is deleted from the engineered cell line using a CRISPR-Cas system.

The engineered cell lines disclosed herein result in an increase in total viral particle production as compared to a control cell line. In certain embodiments, the increase in viral particle production is at least about 10%, such as at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300%, relative to the control cell line. In certain embodiments, the engineered cell line may increase viral particle production by up to about 10%, such as up to about 15%, up to about 20%, up to about 25%, up to about 30%, up to about 35%, up to about 40%, up to about 45%, up to about 50%, up to about 55%, up to about 60%, up to about 65%, up to about 70%, up to about 75%, up to about 80%, up to about 85%, up to about 90%, up to about 95%, up to about 100%, up to about 150%, up to about 200%, up to about 250%, or up to about 300%, as compared to a control cell line. In certain embodiments, the engineered cell line may increase viral particle production by at least 0.3 log, such as at least 0.4 log, at least 0.5 log, at least 0.6 log, at least 0.7 log, at least 0.8 log, at least 0.9 log, at least 1.0 log, at least 1.1 log, at least 1.2 log, at least 1.3 log, at least 1.4 log, or at least 1.5 log, as compared to a control cell line. In certain embodiments, the engineered cell line may increase viral particle production by up to 0.3 log, such as up to 0.4 log, up to 0.5 log, up to 0.6 log, up to 0.7 log, up to 0.8 log, up to 0.9 log, up to 1.0 log, up to 1.1 log, up to 1.2 log, up to 1.3 log, up to 1.4 log, or up to 1.5 log, as compared to a control cell line. In certain embodiments, the engineered cell line may increase viral particle production by 0.5 to 1.5 log or 1.0 to 1.5 log, or about 1.5 log, as compared to a control cell line.

In certain embodiments, there is an increase in the ratio of infectious viral particle release to total viral particle production, as compared to a control cell line. In certain embodiments, the increase in the ratio of infectious viral particle production to total viral particle production is at least about 1%, such as at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75%, relative to the control cell line.

The viral particle may be from any virus known to replicate in a living cell using the cell's reproductive machinery. In certain embodiments, the virus is selected from influenza virus, dengue virus, yellow fever virus, RSV, herpes simplex virus, HIV, hepatitis virus, coronavirus, or a virus from the Rhabdoviridae family, such as rabies virus or VSV. For instance, in certain embodiments, the virus is an influenza virus, such as an influenza A virus or an influenza B virus.

All nomenclature used to classify influenza virus is that commonly used by those skilled in the art. Thus, a Type, or Group, of influenza virus refers to the three main types of influenza: influenza Type A, influenza Type B or influenza Type C that infect humans. Influenza A and B cause significant morbidity and mortality each year. It is understood by those skilled in the art that the designation of a virus as a specific Type relates to sequence difference in the respective Ml (matrix) protein or P (nucleoprotein). Type A influenza viruses are further divided into group 1 and group 2. These groups are further divided into subtypes, which refers to classification of a virus based on the sequences of two proteins on the surface of the virus, hemagglutinin (HA) and neuraminidase (NA). Currently, there are 18 recognized HA subtypes (H1-H18) and 11 recognized NA subtypes (N1-N11). Group 1 contains N1, N4, N5, and N8 and H1, H2, H5, H6, H8, H9, H11, H12, H13, H16, H17 and H18. Group 2 contains N2, N3, N6, N7, and N9 and H3, H4, H7, H10, H14, and H15. While there are potentially 198 different influenza A subtype combinations, only about 131 subtypes have been detected in nature. Current subtypes of influenza A viruses that commonly circulate in the human population, giving rise to seasonal outbreaks, include A (H1N1) and A (H3N2). Influenza B subtypes may include any subtype known in the art, including, for example, an influenza virus strain from a B/Victoria lineage or an influenza virus strain from a B/Yamagata lineage.

Accordingly, the engineered cell lines disclosed herein may comprise a Vero cell or cell line comprising a modification of an ISG15 gene, such as deletion of the ISG15 gene, wherein the modification results in an increase in total influenza virus particle production and/or infection influenza virus particle production as compared to a control Vero cell line.

### Identifying Target Genes

In certain embodiments, disclosed herein are methods of identifying a target gene or genes for deletion in a host cell genome, wherein deletion of the target gene or genes results in enhanced production of viral particles and/or infectious viral particles. In certain embodiments, the host cell is a Vero cell. Target genes may be identified by any method or combination of methods known in the art.

In certain embodiments, disclosed herein are methods of identifying a gene or genes for deletion in a cell or cell line, the method comprising (1) infecting the cell or cell line with a virus; (2) detecting expression levels of multiple genes in the infected cell or cell line and comparing those expression levels to expression levels of the multiple genes in a control cell or cell line that is not infected with the virus; and (3) identify a target gene or genes that are differentially expressed in the infected cell or cell line. In certain embodiments, the method further comprises analyzing the target gene or genes that are differentially expressed to identify one or more gene targets involved in multiple protein-protein networks and selecting at least one differentially expressed gene target for deletion in the cell or cell line, wherein deletion of the at least one differentially expressed gene target increases viral particle production of the virus.

In certain embodiments, target genes are identified through at least one of differential gene expression analysis, GSEA, and/or Network Topology Analysis to identify protein-protein networks. In certain embodiments, all three of differential gene expression analysis, GSEA, and analysis of protein-protein interactions may be used to identify a target gene or genes.

Differential gene expression analysis: Gene expression differences, such as up-regulation or down-regulation, may be evaluated between a host cell infected with a virus and a control host cell that has not been infected with the virus. To determine gene expression differences between infected and non-infected cells, RNA sequencing data from both genomes may be obtained using methods known in the art, such as DESeq2 analysis. Love M. I., et al., Moderated estimation of fold change and dispersion for RNA-seq data with DESeq2, Genome Biology 2014. RNA sequencing data identifies genes that are differentially expressed across multiple groups of samples. Accordingly, in certain embodiments, RNA sequencing data from host cells that have been infected with a virus may be obtained and compared to RNA sequencing data from host cells that have not been infected with a virus or from known normalized gene expression data. In certain embodiments, RNA sequencing data from the infected cell may be obtained at any time period post-infection, including, for example, about 30 minutes post-infection, about 1 hour post-infection (hpi), about 2 hpi, about 4 hpi, about 6 hpi, about 8 hpi, about 10 hpi, about 12 hpi, about 16 hpi, about 20 hpi, about 24 hpi, about 48 hpi, or about 72 hpi.

As used herein, measuring or detecting the expression of any of the foregoing genes or nucleic acids comprises measuring or detecting any nucleic acid transcript (e.g., mRNA, cDNA, or genomic DNA) corresponding to the gene of interest or the protein encoded thereby. If a gene is associated with more than one mRNA transcript or isoform, the expression of the gene can be measured or detected by measuring or detecting one or more of the mRNA transcripts of the gene, or all of the mRNA transcripts associated with the gene.

Typically, gene expression can be detected or measured on the basis of mRNA or cDNA levels, although protein levels also can be used when appropriate. Any quantitative or qualitative method for measuring mRNA levels, cDNA, or protein levels can be used. Suitable methods of detecting or measuring mRNA or cDNA levels include, for example, Northern Blotting, microarray analysis, or a nucleic acid amplification procedure, such as reverse-transcription PCR (RT-PCR), real-time RT-PCR, also known as quantitative RT-PCR (qRT-PCR), and/or digital droplet PCT (ddPCR). Such methods are well known in the art. See e.g., Sambrook et al., Molecular Cloning: A Laboratory Manual, 4^{th} Ed., Cold Spring Harbor Press, Cold Spring Harbor, N.Y., 2012. Other techniques include digital, multiplexed analysis of gene expression, such as the nCounter® (NanoString Technologies, Seattle, WA) gene expression assays, which are further described in US 2010/0112710 and US 2010/0047924.

Detecting a nucleic acid of interest generally involves hybridization between a target (e.g. mRNA, cDNA, or genomic DNA) and a probe. Sequences of many genes are readily known. Therefore, one of skill in the art can readily design hybridization probes for detecting those genes. See, e.g., Sambrook et al., Molecular Cloning: A Laboratory Manual, 4^{th} Ed., Cold Spring Harbor Press, Cold Spring Harbor, N.Y., 2012. Each probe may be substantially specific for its target, to avoid any cross-hybridization and false positives. An alternative to using specific probes is to use specific reagents when deriving materials from transcripts (e.g., during cDNA production, or using target-specific primers during amplification). In both cases specificity can be achieved by hybridization to portions of the targets that are substantially unique within the group of genes being analyzed, for example hybridization to the polyA tail would not provide specificity. If a target has multiple splice variants, it is possible to design a hybridization reagent that recognizes a region common to each variant and/or to use more than one reagent, each of which may recognize one or more variants.

In certain embodiments, microarray analysis or a PCR-based method is used. In this respect, measuring the expression of the foregoing nucleic acids can comprise, for instance, contacting a sample with polynucleotide probes specific to the genes of interest, or with primers designed to amplify a portion of the genes of interest, and detecting binding of the probes to the nucleic acid targets or amplification of the nucleic acids, respectively. Detailed protocols for designing PCR primers are known in the art. See e.g., Sambrook et al., Molecular Cloning: A Laboratory Manual, 4^{th} Ed., Cold Spring Harbor Press, Cold Spring Harbor, N.Y., 2012. Similarly, detailed protocols for preparing and using microarrays to analyze gene expression are known in the art and described herein.

Alternatively or additionally, expression levels of genes can be determined at the protein level, meaning that levels of proteins encoded by the genes discussed herein are measured. Several methods and devices are known for determining levels of proteins including immunoassays, such as described, for example, in U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; 5,458,852; and 5,480,792, each of which is hereby incorporated by reference in its entirety. These assays may include various sandwich, competitive, or non-competitive assay formats, to generate a signal that is related to the presence or amount of a protein of interest. Any suitable immunoassay may be utilized, for example, lateral flow, enzyme-linked immunoassays (ELISA), radioimmunoassays (RIAs), competitive binding assays, and the like. Numerous formats for antibody arrays have been described. Such arrays may include different antibodies having specificity for different proteins intended to be detected. For example, at least 100 different antibodies are used to detect 100 different protein targets, each antibody being specific for one target. Other ligands having specificity for a particular protein target can also be used, such as the synthetic antibodies disclosed in WO 2008/048970, which is hereby incorporated by reference in its entirety. Other compounds with a desired binding specificity can be selected from random libraries of peptides or small molecules. U.S. Pat. No. 5,922,615, which is hereby incorporated by reference in its entirety, describes a device that uses multiple discrete zones of immobilized antibodies on membranes to detect multiple target antigens in an array. Microtiter plates or automation can be used to facilitate detection of large numbers of different proteins.

Although immunoassays have been used for the identification and quantification of proteins, recent advances in mass spectrometry (MS) techniques have led to the development of sensitive, high-throughput MS protein analyses. The MS methods can be used to detect low abundant proteins in complex biological samples. For example, it is possible to perform targeted MS by fractionating the biological sample prior to MS analysis. Common techniques for carrying out such fractionation prior to MS analysis include, for example, two-dimensional electrophoresis, liquid chromatography, and capillary electrophoresis.

Western blotting allows determining specific proteins (native or denatured) from extracts made from cells or tissues, before or after any purification steps. Proteins are generally separated by size using gel electrophoresis before being transferred to a synthetic membrane (typically nitrocellulose or PVDF) via dry, semi-dry, or wet blotting methods. The membrane can then be probed using antibodies using methods similar to immunohistochemistry, but without a need for fixation. Detection is typically performed using peroxidase linked antibodies to catalyze a chemiluminescent reaction. Western blotting is a routine molecular biology method that can be used to semi quantitatively or quantitatively compare protein levels between extracts. The size separation prior to blotting allows the protein molecular weight to be gauged as compared with known molecular weight markers. Western blotting is an analytical technique used to detect specific proteins in a given sample of tissue homogenate or extract. It uses gel electrophoresis to separate proteins by the length of the polypeptide (denaturing conditions) or by the 3-D structure of the protein (native/non-denaturing conditions).

Gene Set Enrichment Analysis: Gene Set Enrichment Analysis (GSEA) may be used to further interpret differential gene expression data. GSEA focuses on gene sets, or groups of genes that share a common biological function, chromosomal location, and/or regulation. Subramanian, A. et al., Gene set enrichment analysis: A knowledge-based approach for interpreting genome-wide expression profiles, Proc. Nat'l Acad. Sci. USA 2005, 102 (43): 15545-15550. In certain embodiments of all aspects of the disclosure, GSEA may be used to identify classes of genes that may have an association with increased viral particle production and/or infectious viral particle production in a cell or cell line. GSEA allows for the interpretation of gene expression data, focusing on gene sets, or groups of genes that share a common function, location, or regulation. Gene sets are available in a searchable format, for example in the electronic Molecular Signatures Database (MSigDB). Genes from a sample may be ranked by differential gene expression, comparing the quantity of viral particles produced in infected and non-infected cells, and used to screen for enriched gene sets in the MSigDB.

In certain embodiments, GSEA may involve calculation of an enrichment score ES representing the extent to which a gene set is over-represented among all of the differentially expressed genes (due to either overexpression or underexpression of the differentially expressed genes). The statistical significance (p-value) of the ES may also be calculated, as well as a normalized enrichment score value (NES); likewise, a false discovery rate (FDR) may be calculated to correspond to the proportion of false positives for any given NES. Subramanian 2005. The NES computed for each gene set may reflect the degree to which a gene set correlates with increased viral particle production. The NES for GSEA may be calculated by any means known in the art, including, for example, using Reactome (Croft, D et al., The Reactome pathway knowledgebase, Nucleic Acids Res. 2014, 42: D472-D477) and/or WebGestalt (WEB-based Gene SeT Analysis Toolkit) (Liao, Y. et al., WebGestalt 2019: gene set analysis toolkit with revamped UIs and APIs, Nucleic Acids Res. 2019, 47 (W1): W199-W205). In certain embodiments, a NES for a gene set may be a positive value, indicating upregulated expression of the genes in the gene set, and be greater than about 0.5, such as greater than about 1.0, greater than about 1.5, greater than about 2.0, or greater than about 2.5. In certain embodiments, a NES for a gene set may a negative value, indicating down-regulated expression of the genes in the gene set, and be less than about −0.5, such as less than about −1.0, less than about −1.5, less than about −2.0, or less than about −2.5.

In certain embodiments, hallmark gene pathways may be identified through the use of GSEA as disclosed herein. In certain embodiments, at least one of the following gene pathways may be up-regulated in a cell or cell line after infection with an influenza virus: G-protein Coupled Receptor (GPCR) ligand binding, Signaling by GPCR, Class A/1 (Rhodopsin-like receptors), GPCR downstream signaling, G alpha (i) signaling events, interferon alpha/beta signaling, synthesis of DNA, visual phototransduction, peptide ligand-binding receptors, DNA replication, Orc1 removal from chromatin, assembly of the pre-replicative complex, DNA replication pre-initiation, mitotic metaphase and anaphase, mitotic anaphase, G2/M checkpoints, switching of origins to a post-replicative state, antiviral mechanism by IFN-stimulated genes, separation of sister chromatids, and cross-presentation of soluble exogenous antigens (endosomes).

In certain embodiments, at least one of the following gene pathways may be upregulated in a cell or cell line after infection with an VSV virus: interferon alpha/beta signaling, interferon signaling, GPCR ligand binding, antiviral mechanism by IFN-stimulated genes, class A/1 (Rhodopsin-like receptors), peptide ligand-binding receptors, interleukin-10 signaling, signaling by GPCR, chemokine receptors bind chemokines, cytokine signaling in immune system, interferon gamma signaling, GPCR downstream signaling, ISG15 antiviral mechanism, G alpha (i) signaling events, class B/2 (Secretin family receptors), OAS antiviral response, SLC transporter disorders, cholesterol biosynthesis, DDX58/IFIH1-mediated inductions of interferon-alpha/beta, and activation of matrix metalloproteinases.

In certain embodiments, at least one of the following gene pathways may be up-regulated in a cell infected after infection with a virus: GPCR ligand binding, signaling by GPCR, class A/1 (Rhodopsin-like receptors), GPCR downstream signaling, G alpha (i) signaling events, interferon alpha/beta signaling, peptide ligand-binding receptors, and antiviral mechanism by IFN-stimulated genes.

In certain embodiments, at least one of the following gene pathways may be down-regulated in a cell or cell line after infection with an influenza virus: selenocysteine synthesis, major pathway of rRNA processing in the nucleolus and cytosol, eukaryotic translation termination, NMD independent of the EJC, peptide chain elongation, rRNA processing, NMD enhanced by the EJC, NMD, eukaryotic translation elongation, formation of a pool of free 40S subunits, L13a-mediated translational silencing of Ceruloplasmin expression, GTP hydrolysis and joining of the 60S ribosomal subunit, signaling by non-receptor tyrosine kinases, signaling by PTK6, cap-dependent translation initiation, eukaryotic translation initiation, signaling by NTRK2 (TRKB), viral mRNA translation, signaling by ERBB2, and selenoamino acid metabolism.

In certain embodiments, at least one of the following gene pathways may be down-regulated in a cell or cell line after infection with an VSV virus: rRNA processing in the nucleus and cytosol, eukaryotic translation elongation, major pathway of rRNA processing in the nucleolus and cytosol, rRNA processing, NMD independent of the EJC, peptide chain elongation, L13a-mediated translational silencing of ceruloplasmin expression, cap-dependent translation initiation, eukaryotic translation initiation, eukaryotic translation termination, selenocysteine synthesis, GTP hydrolysis and joining of the 60S ribosomal subunit, Formation of a pool of free 40S subunits, NMD enhanced by the EJC, NMD, viral mRNA translation, selenoamino acid metabolism, activation of the mRNA upon binding of the cap-binding complex and eIFs, translation initiation complex formation, and influenza viral RNA transcription and replication.

In certain embodiments, at least one of the following gene pathways may be down-regulated in a cell infected after infection with a virus: selenocysteine synthesis, major pathway of rRNA processing in the nucleolus and cytosol, eukaryotic translation termination, NMD independent of the EJC, peptide chain elongation, rRNA processing, NMD enhanced by the EJC, NMD, eukaryotic translation elongation, formation of a pool of free 40S subunits, L13a-mediated translational silencing of Ceruloplasmin expression, GTP hydrolysis and joining of the 60S ribosomal subunit, cap-dependent translation initiation, eukaryotic translation initiation, viral mRNA translation, selenoamino acid metabolism.

In certain embodiments disclosed herein, the method of identifying a gene for modification in a cell or cell line comprises analyzing the genes identified through differential expression and GSEA and does not further comprise analyzing the gene targets involved in multiple protein-protein interactions. In certain embodiments, however, potential target genes may be further identified by combining the results of differential expression and/or GSEA with a network topology analysis, such as analyzing multiple protein-protein interactions to identify a target gene or genes for modification in a cell, wherein modification of the target gene or genes increases viral particle production.

Network Topology Analysis: In certain embodiments, differentially expressed genes and gene sets identified through GSEA may be further narrowed through the use of a network topology analysis to identify target genes involved in certain protein-protein interaction (PPI) networks. In certain embodiments, a list of target genes may be generated and/or filtered based on the Network Retrieval and Prioritization construction method, as described, for example, in Wang, J. et al., Proteome Profiling Outperforms Transcriptome Profiling for Coexpression Based Gene Function Prediction, Mol Cell Proteomics 2017, 16 (1): 121-134. For example, in certain embodiments, a random walk analysis may be used to calculate random walk probability for a given gene or genes (known as seeds). Then, relationships among those seeds may be identified in a selected network to arrive at a retrieval sub-network, wherein gene with a high level of random walk probability may be selected. A Network Topology Analysis may use random walk-based network propagation by identifying genes that are potentially biologically significant, and each gene in a PPI network may be attributed a score, wherein the statistical significance of the score can be calculated via two p-values: a global p-value (which significance is the result of a non-random association between the gene in the PPI network and the input seeds), and a local p-value (which significance indicates that the gene did not acquire a significant association with the input seeds only due to network topology).

Enrichment analysis of the retrieved sub-networks may then by conducted using any means known in the art, including, for example, the PPI BIOGRID database (Stark C. et al., BioGRID: a general repository for interaction datasets, Nucleic Acids Res. 2006, 34: D535-539) and Gene Ontology (GO) Biology Process terms (Harris, M. A. et al., The Gene Ontology (GO) database and informatics resource, Nucleic Acids Res. 2004, 32: D258-D261. As is known in the art, the GO terms provide a systematic language for the description of genes and gene products in three separate domains that are shared by all organisms: molecular function, biological process, and cellular components. GO terms may be used, for example, for gathering functional and biological significance from large datasets, such as those that may result, for example, from differential gene expression analysis and GSEA.

In certain embodiments disclosed herein, gene targets, such as those identified, for example, through differential gene expression and GSEA, may be further analyzed to identify one or more gene targets involved in multiple protein-protein networks. For example, in certain embodiments, a single gene target may share at least two GO pathways. In certain embodiments, the multiple protein-protein networks comprise at least two of the following: defense response (GO: 0006952), response to virus (GO: 0009615), viral genome replication (GO: 0019079), response to cytokine (GO: 0034097), response to type I interferon (GO: 0034340), regulation of viral genome replication (GO: 0045069), defense response to virus (GO: 0051607), cell death (GO: 0008219), viral life cycle (GO: 0019058), negative regulation of viral genome replication (GO: 0045071), and cellular response to cytokine stimulus (GO: 0071345). In certain embodiments, the multiple protein-protein networks comprise at least two of the following: defense response, response to virus, viral genome replication, response to cytokine, response to type 1 interferon, regulation of viral genome replication, defense response to virus, negative regulation of viral genome replication, and cellular response to cytokine stimulus. In certain embodiments, the multiple protein-protein networks comprise at least two of the following: response to virus, viral genome replication, response to type 1 interferon, and defense response to virus.

In certain embodiments of the methods of identifying a gene or genes for deletion disclosed herein, the cell or cell line may be any host cell or cell line known for propagation of viral particles, for example for the production of viral vaccines. In certain embodiments, the cell or cell line is a primate, such as monkey (e.g., Vero cell line) or human, canine, bovine, porcine, feline, murine, hamster, or rabbit, for example. In certain embodiments, the cell or cell line is a Vero cell line. In certain embodiments, the cell or cell line is a Madin-Darby Canine (MDCK) cell, and in certain embodiments, the cell or cell line is a Human Embryonic Kidney (HEK) cell.

In certain embodiments of the methods of identifying a gene or genes for deletion disclosed herein, the virus may be any virus known to replicate in a cell or cell line, including, but not limited to, a Vero cell line. For example, in certain embodiments, the virus is selected from influenza virus (such as influenza A virus or influenza B virus), dengue virus, yellow fever virus, RSV, herpes simplex virus, HIV, hepatitis virus, coronavirus, or a virus from the Rhabdoviridae family, such as rabies virus or VSV.

After identification of the gene or genes for modification in a cell or cell line, the methods disclosed herein may further comprise modifying or deleting said gene or genes, or decreasing expression of said gene or genes, by any method known in the art. In certain embodiments, the identified gene or genes is deleted from the cell or cell line using a CRISPR-Case system, as disclosed above.

### Methods of Using Engineered Cell Lines

The engineered cell lines disclosed herein may be used, for example, to increase viral particle production. In certain embodiments, disclosed herein is a method of increasing viral particle production. In the methods disclosed herein, an engineered cell line may be infected with a virus and incubated under conditions suitable for production of the virus by the engineered cell line; thereafter, the virus produced by the engineered cell line may be harvested. Harvested viral particles may then be used, for example, for further research and/or for production of vaccine compositions. In certain embodiments, the engineered cell line comprises a modification of at least one gene resulting in an increase in total viral particle production and/or infectious viral particle production as compared to a control cell line that is identical to the engineered cell line expect for the modification.

In certain embodiments, the modification is in at least one of the following genes: APOA1, CCL2, CCL5, CYP19A1, CXCL8, ELF3, FOS, HERC3, HERC5, IFIT1, IFIT2, IFIT3, IRF7, ISG15, KRT15, KRT19, MX1, NGFR, PTGS2, PTPN6, RET, ROS1, SFRP1, SOX2, SPP1, TNF, TNFRSF4, TRAF1, and VAV3. In certain embodiments, the modification is in at least one of the following genes: CCL2, CCL5, CXCL8, HERC5, IFIT1, IFIT2, IFIT3, and ISG15. In certain embodiments, the modification is in an ISG15 gene. The modification may be a modification to decrease expression of the at least one gene. For example, in certain embodiments, the modification results in the deletion of the at least one gene, such as deletion by a CRISPR-Cas system.

In certain embodiments, the engineered cell line may increase viral particle production by at least about 20%, such as at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300%, as compared to a control cell line. In certain embodiments, the engineered cell line may increase viral particle production by about 20% to about 300%, about 50% to about 300%, or about 100% to about 300%, as compared to a control cell line. In certain embodiments, the engineered cell line may increase viral particle production by up to 1.5 log, including, for example, an increase of 0.5 to 1.5 log or 1.0 to 1.5 log, or about 1.5 log, as compared to a control cell line. The viral particle production may be measured by any means known in the art for counting viral genomes. For example, in certain embodiments, the viral particle production is measured via Median Tissue Culture Infectious Dose (TCID₅₀) assay, hemagglutination assay, or PCR, such as ddPCR or qRT-PCR.

The number of infectious virus particles may also be quantified by any means known in the art. In certain embodiments, the number of infectious viral particles is quantified by using the TCID₅₀ assay and/or a plaque assay. Both the TCID₅₀ assay and the plaque assay work by adding a serial dilution of the virus sample to cells, for example in a 96 well plate format. The type of cell is specifically selected to show a cytopathic effect (CPE), i.e., morphological changes upon infection with the virus or cell death. After an incubation period, the cells are inspected for CPE or cell death, and each well is classified as infected or not infected. Colorimetric or fluorometric readouts are also possible, which can increase assay sensitivity. The dilution at which 50% of the wells show a CPE is used to calculate the TCID₅₀ of the virus sample. This calculation can generally be done by a variety of mathematical approaches, e.g., the Spearman-Karber method or the Reed-Muench method. Virus titer is expressed as TCID₅₀/mL. For plaque assay quantification, the number of plaques that are formed by a virus at varying dilutions may be quantified per well, i.e., the number of plaque-forming units (PFUs) may be quantified per well. The log titre may be expressed as the log of PFU/mL. A comparison of the TCID₅₀ assay and the plaque assay is discussed, for example, in Smither, S. J. et al., Comparison of the plaque assay and 50% tissue culture infectious dose assay as methods for measuring filovirus infectivity, J. Virological Methods 2013, 193 (2): 565-71.

An hemagglutinin assay applies the process of hemagglutination, in which sialic acid receptors on the surface of red blood cells (RBCs) bind to a hemagglutinin glycoprotein found on the surface of a virus, such as the influenza virus, and create a network, or lattice structure, of interconnected RBCs and virus particles, referred to as hemagglutination, which occurs in a concentration dependent manner on the virus particles. One goal of an hemagglutinin assay can be to characterize the concentration of viral particles relative to their ability to elicit hemagglutination in the assay.

PCR techniques may also be used to amplify and quantify viral genomes (i.e., DNA or RNA). In certain embodiments, quantification by PCR comprises multiple serial dilution of samples of unknown concentration in parallel with samples of known concentration for reference and calibration. Quantification may be achieved, for example, using a wide variety of known fluorescence detection strategies. One method of PCR includes ddPCR, which is a form of digital PCR relying on water-oil emulsion droplet technology. In certain embodiments, a sample may be fractionated into thousands of droplets, such that PCR amplification of the target nucleic acid occurs within each individual droplet. Viral particles may then be quantified, for example, in terms of Vg/mL.

As is known in the art, PCR amplifies all target nucleic acid material, including nucleic acid originating from both intact infectious viral particles and defective viral particles, as well as free nucleic acid. Accordingly, PCR results, which may be expressed in terms of viral genome (Vg)/mL, are often higher in quantity than virus titer (e.g., TCID₅₀/mL) results. Accordingly, in addition to measuring viral particle production, for example by PCR, one may also measure infectious viral particle production, for example by TCID₅₀, and then compare the ratio between the two values. In certain embodiments of the engineered cell lines and methods disclosed herein, the ratio of infectious viral particle production to total viral particle production is at least about 3%, such as at least about 5%, at least about 15%, at least about 20%, at least about 25%, at least about 30%, at least about 35%, at least about 40%, at least about 45%, at least about 50%, at least about 55%, at least about 60%, at least about 65%, or at least about 70%.

### Vaccine Compositions

Also disclosed herein are methods of using the engineered cell lines to produce viral particles for use in a vaccine composition. For example, in certain embodiments, after harvesting the virus produced by the engineered cell line, the virus, which may be a live virus, a live attenuated virus, or an inactivated virus, may be added to a vaccine composition. For example, certain known licensed influenza vaccine compositions are inactivated vaccines, containing entire virions or virions subjected to treatment with agents that dissolve lipids (“split” vaccines), purified glycoproteins expressed in cell culture (“sub-unit vaccines”), or live attenuated virus vaccines.

In certain embodiments, disclosed herein are vaccine compositions comprising virus particles harvested from an engineered cell line, wherein the engineered cell line comprises a modification in one or more genes, such as ISG15, wherein the modification in the one or more gene results in an increase in total viral particle production and/or infectious viral particle production as compared to a control cell line that is identical to the engineered cell line except for the modification in the one or more genes.

The vaccine composition can also further comprise an adjuvant. As used herein, the term “adjuvant” refers to a substance or vehicle that non-specifically enhances the immune response to an antigen. Adjuvants can include a suspension of minerals (alum, aluminum salts, including, for example, aluminum hydroxide/oxyhydroxide (AlOOH), aluminum phosphate (AlPO₄), aluminum hydroxyphosphate sulfate (AAHS) and/or potassium aluminum sulfate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as lipids and costimulatory molecules. Exemplary biological adjuvants include AS04 (Didierlaurent, A. M. et al, AS04, an Aluminum Salt- and TLR4 Agonist-Based Adjuvant System, Induces a Transient Localized Innate Immune Response Leading to Enhanced Adaptive Immunity, J. IMMUNOL. 2009, 183:6186-6197), IL-2, RANTES, GM-CSF, TNF-α, IFN-γ, G-CSF, LFA-3, CD72, B7-1, B7-2, OX-40L and 41 BBL.

In addition to the viral particles, and an optional adjuvant, the vaccine composition may also further comprise one or more pharmaceutically acceptable excipients. In general, the nature of the excipient will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, vaccine compositions to be administered can contain minor amounts of non-toxic auxiliary substances, such as wetting or emulsifying agents, pharmaceutically acceptable salts to adjust the osmotic pressure, preservatives, stabilizers, buffers, sugars, amino acids, and pH buffering agents and the like, for example sodium acetate or sorbitan monolaurate.

Typically, the vaccine composition is a sterile, liquid solution formulated for parenteral administration, such as intravenous, subcutaneous, intraperitoneal, intradermal, or intramuscular. The vaccine composition may also be formulated for intranasal or inhalation administration. The vaccine composition can also be formulated for any other intended route of administration.

In some embodiments, a vaccine composition is formulated for intradermal injection, intranasal administration or intramuscular injection. In some embodiments, injectables are prepared in conventional forms, either as liquid solutions or suspensions, solid forms suitable for solution or suspension in liquid prior to injection, or as emulsions. In some embodiments, injection solutions and suspensions are prepared from sterile powders or granules. General considerations in the formulation and manufacture of pharmaceutical agents for administration by these routes may be found, for example, in Remington's Pharmaceutical Sciences, 19^{th} ed., Mack Publishing Co., Easton, PA, 1995; incorporated herein by reference. At present the oral or nasal spray or aerosol route (e.g., by inhalation) are most commonly used to deliver therapeutic agents directly to the lungs and respiratory system. In some embodiments, the vaccine composition is administered using a device that delivers a metered dosage of the vaccine composition. Suitable devices for use in delivering intradermal pharmaceutical compositions described herein include short needle devices such as those described in U.S. Pat. Nos. 4,886,499, 5,190,521, 5,328,483, 5,527,288, 4,270,537, 5,015,235, 5,141,496, 5,417,662 (all of which are incorporated herein by reference). Intradermal compositions may also be administered by devices which limit the effective penetration length of a needle into the skin, such as those described in WO1999/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO1997/37705, and WO1997/13537 (all of which are incorporated herein by reference). Also suitable are ballistic powder/particle delivery devices which use compressed gas to accelerate vaccine in powder form through the outer layers of the skin to the dermis. Additionally, conventional syringes may be used in the classical mantoux method of intradermal administration.

Preparations for parenteral administration typically include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The present disclosure will be more fully understood by reference to the following Examples.

### Examples

The following examples are to be considered illustrative and not limiting on the scope of the disclosure described above.

Cell lines and culture media: The Vero WHO cell line disclosed in the following Examples was at passage 138. This cell line was derived from a vial of Vero ATCC CCL-81 that was sent to WHO at passage 124 for analysis and establishment of the Vero WHO master cell bank approved for vaccine production. The cells were grown in static culture at 37° C. and 5% CO₂ in a humidified incubator (Infors HT, Switzerland). Cells were passaged twice weekly using TrypLE® Express (Thermo Fisher Scientific) as a dissociation reagent. A serum-free adapted sub-cell line grown in OptiPRO® medium (Thermo Fisher Scientific) supplemented with 4 mM GlutaMAX® (Thermo Fisher Scientific) was cryopreserved at a passage number of 151 in OptiPRO® medium supplemented with 4 mM GlutaMAX® and 10% DMSO (Sigma, USA).

ISG15−/− Vero cell lines were received and thawed for 5 passages before being cryopreserved. The cells were then thawed again and continued passaging for 4 more passages (“p5+4”) or continuously passaged from the initial thaw for 17 passages (“p17”). The cell line was derived from a vial of Vero ATCC CCL-81 that was sent to WHO at passage 124 for analysis and establishment of the Vero WHO master cell bank approved for vaccine production. ISG15−/− Vero cells and control cell lines were cultured in serum-free, ultra-low protein media containing no proteins, peptides, or other components of animal or human origin (VP-SFM AGT™ from Thermo Fisher Scientific), using pharmaceutical grade reagents and equipment.

### Example 1—Kinetic Analysis and Infection of Cells

Initially, a quality control kinetics experiment was run in which Vero cells were infected with influenza virus A (IVA) Puerto Rico 8 strain or rVSV-GFP at a multiplicity of infection (MOI) of 10 to quantify through time the viral production rate and the cells' viability. The results led to the deduction of the best time to harvest samples for RNA sequencing (e.g., the highest viability before induction of cell death).

Supernatant was harvested at several time points, and cell viability was monitored. In order to quantify the virus production for each time point, TCID₅₀ and hemagglutination assays were used to quantify viral particles and infectious viral particles (for IVA) and infectious viral particles (for rVSV-GFP).

The optimal harvesting time point for RNA sequencing was selected for IVA and rVSV based on the infectious viral particles' production level and the viability of the cells. Thus, for IVA, the selected time points were determined to be at relatively early stages of infection (4 hours post infection (hpi)) and at the peak of infectious viral particles production (24 hpi). For rVSV-GFP, given the apparition of cytopathic effects at the early stages of infection (8 hpi), the time points selected were 2 hpi and 6 hpi to ensure that pathways such as cell death were not falsely enriched due to sample quality issues.

Based on the results of the kinetics analysis, for further transcriptome analysis, Vero WHO cells at passage 153 were infected with either IVA Puerto Rico 8 or rVSV-GFP at an MOI of 10. The IVA infected cells were harvested at 4 hpi and 24 hpi, and the rVSV-GFP infected cells were harvested at 2 hpi and 6 hpi. The samples were harvested using TrypLER Express and centrifuged at 300×g for 5 minutes. Cell pellets of around 6 million cells were lysed and quickly frozen in a mixture of dry ice/ethanol and stored at −80° C. until further analysis. Samples from non-infected cells were also prepared and sent to sequencing as a control batch. All of the samples were generated in triplicate.

### Example 2—Functional Genomic Analysis and Selection of Target Genes

Identification of differentially expressed genes: Total RNA sequencing (TrueSeq) was performed using Illumina NovaSeq6000 Sprime v1.5, PE100. Following standard quality control, the reads were first aligned to the Vero cell genome, as published in Sène, M.-A. et al., Haplotype-resolved de novo assembly of the Vero cell line genome, NPJ Vaccine 2021, 6 (1): 106, using STAR alignment as describe in Dobin, A. et al., STAR: ultrafast universal RNA-seq aligner, Bioinformatics 2013, 29 (1): 15-21. The resulting BAM files were sorted by name using SAMtools (Li, H. et al., The Sequence Alignment/Map format and SAMtools, Bioinformatics 2009, 25 (16): 2078-2079) before read count. Transcripts were quantified using featureCounts (Liao, Y. et al., featureCounts: an efficient general purpose program for assigning sequence reads to genomic features, Bioinformatics 2014, 30 (7) 923-930). Differential expression analysis of the raw read counts was done using DESeq2, and quality control graphs were produced using DESeq2 and R package, as described in Love, M. I. et al., Moderated estimation of fold change and dispersion for RNA-seq data with DESeq2, Genome Biology 2014, 15:550. The resulting differentially expressed (DE) gene list was filtered with a p-value cut-off of 0.0001.

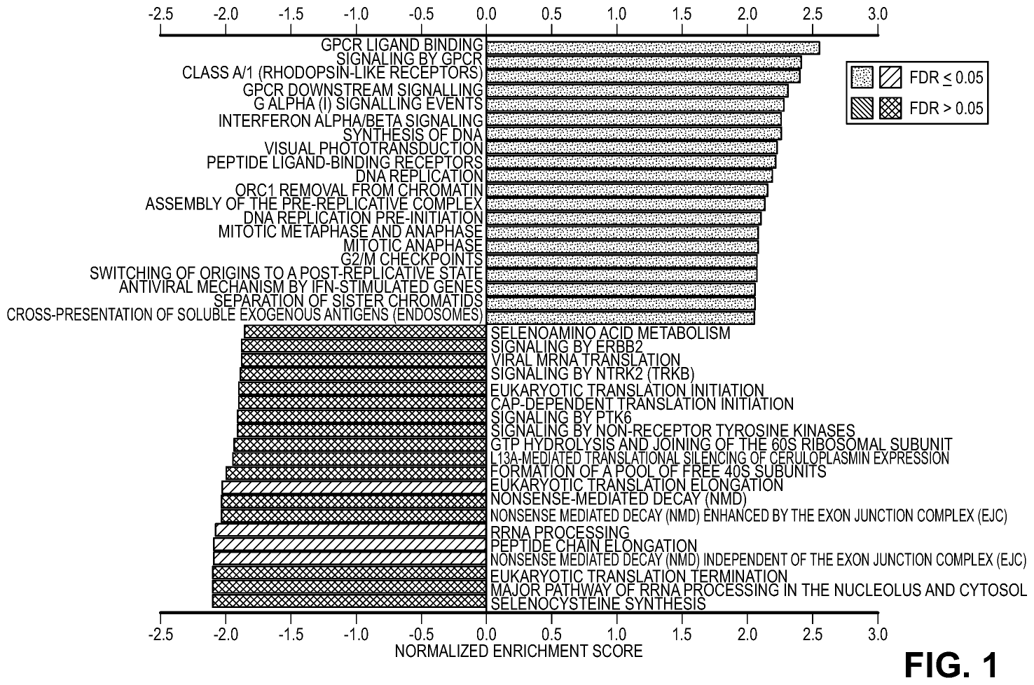

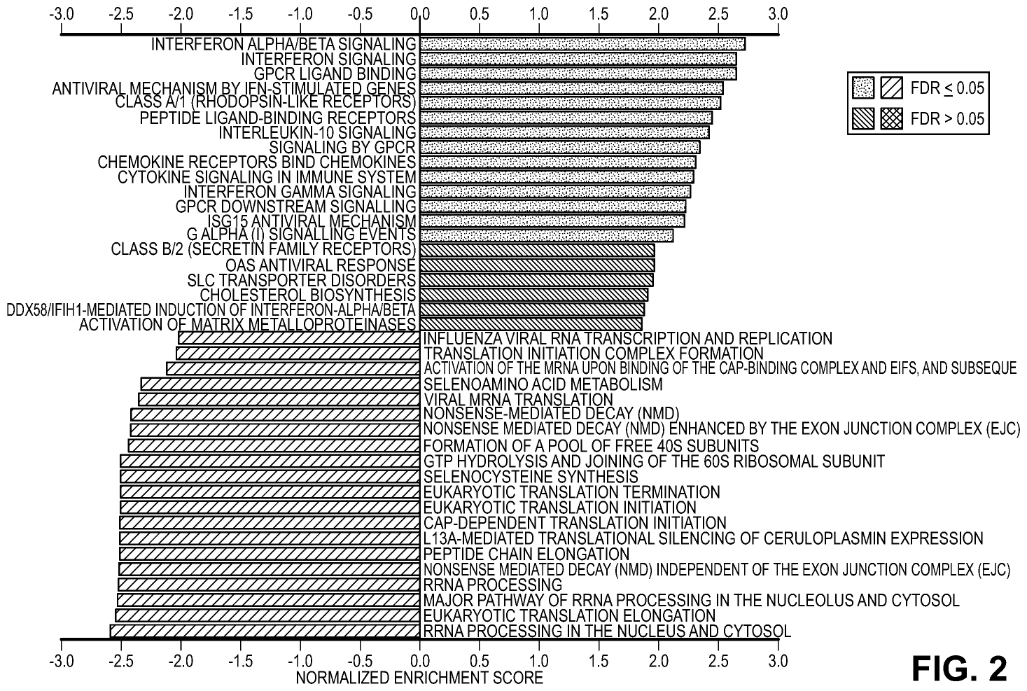

Gene set enrichment analysis: The DE genes were then ranked based on their log 2 fold change. The WebGestalt (WEB-based GEne SeT Analysis Toolkit) was used for gene set enrichment analysis with the Reactome gene set collection (Croft, 2014). To find differentially expressed pathways of genes between adherent and suspension cell lines, gene sets were filtered, and the top 20 gene sets with an adjusted p-value lower than 0.05 were considered as significantly changed. The results are shown below in Table 2 and in FIG. 1 for 24 hpi IVA and in Table 3 and FIG. 2 for rVSV-GFP.

TABLE 2
GSEA of Significantly Enriched Hallmark Pathways for 24 hpi IVA

| Hallmark pathway | FDR |
|---|---|
| GPCR ligand binding | ≤0.05 |
| Signaling by GPCR | ≤0.05 |
| Class A/1 (Rhodopsin-like receptors) | ≤0.05 |
| GPCR downstream signaling | ≤0.05 |
| G alpha (i) signaling events | ≤0.05 |
| Interferon alpha/beta signaling | ≤0.05 |
| Synthesis of DNA | ≤0.05 |
| Visual phototransduction | ≤0.05 |
| Peptide ligand-binding receptors | ≤0.05 |
| DNA replication | ≤0.05 |
| Orc1 removal from chromatin | ≤0.05 |
| Assembly of the pre-replicative complex | ≤0.05 |
| DNA replication pre-initiation | ≤0.05 |
| Mitotic metaphase and anaphase | ≤0.05 |
| Mitotic anaphase | ≤0.05 |
| G2/M checkpoints | ≤0.05 |
| Switching of origins to a post-replicative state | ≤0.05 |
| Antiviral mechanism by IFN-stimulated genes | ≤0.05 |
| Separation of sister chromatids | ≤0.05 |
| Cross-presentation of soluble exogenous antigens | ≤0.05 |
| (endosomes) | |
| Selenoamino acid metabolism | >0.05 |
| Signaling by ERBB2 | >0.05 |
| Viral mRNA translation | >0.05 |
| Signaling by NTRK2 (TRKB) | >0.05 |
| Eukaryotic translation initiation | >0.05 |
| Cap-dependent translation initiation | >0.05 |
| Signaling by PTK6 | >0.05 |
| Signaling by non-receptor tyrosine kinases | >0.05 |
| GTP hydrolysis and joining of the 60S ribosomal subunit | >0.05 |
| L13a-mediated translational silencing of Ceruloplasmin | >0.05 |
| expression | |
| Formation of a pool of free 40S subunits | >0.05 |
| Eukaryotic translation elongation | ≤0.05 |
| Nonsense-mediated decay (NMD) | >0.05 |
| NMD enhanced by the Exon Junction Complex (EJC) | >0.05 |
| rRNA processing | ≤0.05 |
| Peptide chain elongation | ≤0.05 |
| NMD independent of the EJC | ≤0.05 |
| Eukaryotic translation termination | >0.05 |
| Major pathway of rRNA processing in the nucleolus and | >0.05 |
| cytosol | |
| Selenocysteine synthesis | >0.05 |

TABLE 3
GSEA of Significantly Enriched Hallmark Pathways for 6 hpi rVSV-GFP

| Hallmark pathway | FDR |
|---|---|
| Interferon alpha/beta signaling | ≤0.05 |
| Interferon signaling | ≤0.05 |
| GPCR ligand binding | ≤0.05 |
| Antiviral mechanism by IFN-stimulated genes | ≤0.05 |
| Class A/1 (Rhodopsin-like receptors) | ≤0.05 |
| Peptide ligand-binding receptors | ≤0.05 |
| Interleukin-10 signaling | ≤0.05 |
| Signaling by GPCR | ≤0.05 |
| Chemokine receptors bind chemokines | ≤0.05 |
| Cytokine signaling in immune system | ≤0.05 |
| Interferon gamma signaling | ≤0.05 |
| GPCR downstream signaling | ≤0.05 |
| ISG15 antiviral mechanism | ≤0.05 |
| G alpha (i) signaling events | ≤0.05 |
| Class B/2 (Secretin family receptors) | >0.05 |
| OAS antiviral response | >0.05 |
| SLC transporter disorders | >0.05 |
| Cholesterol biosynthesis | >0.05 |
| DDX58/IFIH1-mediated inductions of interferon-alpha/beta | >0.05 |
| Activation of Matrix Metalloproteinases | >0.05 |
| Influenza viral RNA transcription and replication | ≤0.05 |
| Translation initiation complex formation | ≤0.05 |
| Activation of the mRNA upon binding of the cap-binding | ≤0.05 |
| complex and eIFs | |
| Selenoamino acid metabolism | ≤0.05 |
| Viral mRNA translation | ≤0.05 |
| NMD | ≤0.05 |
| NMD enhanced by the EJC | ≤0.05 |
| Formation of a pool of free 40S subunits | ≤0.05 |
| GTP hydrolysis and joining of the 60S ribosomal subunit | ≤0.05 |
| Selenocysteine synthesis | ≤0.05 |
| Eukaryotic translation termination | ≤0.05 |
| Eukaryotic translation initiation | ≤0.05 |
| Cap-dependent translation initiation | ≤0.05 |
| L13a-mediated translational silencing of Ceruloplasmin | ≤0.05 |
| expression | |
| Peptide chain elongation | ≤0.05 |
| NMD independent of the EJC | ≤0.05 |
| rRNA processing | ≤0.05 |
| Major pathway of rRNA processing in the nucleolus and | ≤0.05 |
| cytosol | |
| Eukaryotic translation elongation | ≤0.05 |
| rRNA processing in the nucleus and cytosol | ≤0.05 |

As shown in FIG. 1, for IVA infection at 24 hpi, gene set enrichment analysis (GSEA) using Reactome as a gene set showed a down regulation of major RNA processing gene sets, including the L13a-mediated translational silencing of Ceruloplasmin expression, which correlates with viral evasion strategies via cap snatching and the host cell's attempts to counter that evasion. Selenium related pathways were also down-regulated, including selenoamino acid metabolism and selenocysteine synthesis. See FIG. 1. Indeed, Guillin, O. M. et al., Selenium, Selenoproteins and Viral Infection, Nutrients 2019, 11 (9): 2101 previously showed that selenium and selenoprotein deficiency leads to increased host susceptibility to viral infection. Meanwhile, key immune response related pathways were significantly upregulated, including interferon signaling. An upregulation of IFN-stimulated genes was also observed.

Similar to IVA, rVSV-GFP interaction with Vero cells at 6 hpi showed a down regulation of one of the key quality control mechanisms of RNA processing: Nonsense Mediated Decay (NMD), thus promoting viral reproduction alongside the down regulation of eukaryotic translation elongation. See FIG. 2. Moreover, previously identified antiviral pathways related to interferons were also upregulated in the case of rVSV-GFP infection, notably, the antiviral mechanism by IFN-stimulated genes such as ISG15.

Network Topology Analysis: In order to go beyond gene sets and pathways and identify antiviral genes involved in protein-protein interaction (PPI) networks, a Network Topology Analysis was done for the previously-identified significantly upregulated genes, including 130 genes for IVA 4hpi, 264 genes for IVA 24hpi, and 235 genes for rVSV-GFP 6hpi.

The upregulated part of the gene list generated by DESeq2 was filtered to consider genes with a |log 2 fold change|>2 for Network Topology Analysis (NTA) based on the Network Retrieval & Prioritization construction method by first using random walk analysis to calculate random walk probability for the input gene IDs (seeds), and then identifying the relationships among the seeds in the selected network to return a retrieval sub-network, wherein the top 20 genes with the top random walk probability were highlighted. Indeed, assuming a tight connection between mechanistically important genes and a random distribution of other genes on the network, the NTA uses random walk-based network propagation by identifying those genes that are potentially biologically significant. The input gene IDs (upregulated genes previously filtered) were used as seeds and, based on their overall proximity (as quantified by the random walk similarity) to the input seeds, each gene in the PPI network was attributed a score. Then the statistical significance of those scores was calculated via two p-values: a global p-value, which significance is the result of a non-random association between the gene in the PPI network and the input seeds; and a local p-value, which significance ensures that the gene did not acquire a significant association with the input seeds simply because of network topology.

Finally, enrichment analysis of the retrieved sub-networks was done using the protein-protein interaction (PPI) BIOGRID database and Gene Ontology (GO) Biology Process terms (Stark C. et al., BioGRID: a general repository for interaction datasets, Nucleic Acids Res. 2006, 34: D535-539; Harris, M.A. et al., The Gene Ontology (GO) database and informatics resource, Nucleic Acids Res. 2004, 32: D258-D261). The GO terms were first ranked based on their adjusted p-value, and the top 10 highly significant terms with an adjusted p-value cut-off of 0.01 were considered. The pathways identified and the top-ranking genes are shown below in Table 4.

### Table 4—Upregulated Networks and Associated Genes

| Virus | Pathway GO ID | Pathway GO Name | Top-ranking associated genes |
|---|---|---|---|
| IVA, 4 hpi | GO:0006952 | Defense response | ISG15, IFIT1, IFIT2, IFIT3, |
| | | | HERC5, CCL2, CCL5, CXCL8, |
| | | | FOS, CYP19A1 |
| | GO:0009615 | Response to virus | ISG15, IFIT1, CCL2, CCL5, |
| | | | CXCL8 |
| | GO:0019079 | Viral genome | ISG15, IFIT1, CCL2, CCL5, |
| | | replication | CXCL8 |
| | GO:0034097 | Response to cytokine | ISG15, IFIT1, IFIT2, IFIT3, |
| | | | CCL2, CCL5, CXCL8, FOS, |
| | | | TRAF1 |
| | GO:0034340 | Response to type 1 | ISG15, IFIT1, IFIT2, IFIT3 |
| | | interferon | |
| | GO:0045069 | Regulation of viral | ISG15, IFIT1, CCL5, CXCL8 |
| | | genome replication | |
| | GO:0051607 | Defense response to | ISG15, IFIT1, IFIT2, IFIT3, |
| | | virus | HERC5 |
| IVA, 24 hpi | GO:0006952 | Defense response | ISG15, IFIT1, IFIT2, IFIT3, |
| | | | MX1, ELF3, HERC5, CCL5, |
| | | | CCL2, CXCL8, TNF, PTPN6, |
| | | | TNFRSF4, APOA1, SPP1, IRF7 |
| | GO:0008219 | Cell death | IFIT2, IFIT3, MX1, CCL2, |
| | | | CCL5, KRT15, KRT19, TNF, |
| | | | TRAF1, VAV3, PTPN6, ROS1, |
| | | | NGFR, TNFRSF4, IRF7, RET, |
| | | | SOX2 |
| | GO:0009615 | Response to virus | ISG15, IFIT1, IFIT2, IFIT3, |
| | | | MX1, HERC3, CCL5, TNF, |
| | | | NGFR |
| | GO:0019058 | Viral life cycle | ISG15, IFIT1, MX1, CCL2, |
| | | | CCL5, CXCL8, TNF, TNFRSF4 |
| | GO:0019079 | Viral genome | ISG15, IFIT1, MX1, CCL5, |
| | | replication | CCL2, CXCL8, TNF |
| | GO:0034097 | Response to cytokine | ISG15, IFIT1, IFIT2, IFIT3, |
| | | | MX1, CCL5, CCL2, CXCL8, |
| | | | TNF, TRAF1, PTPN6, |
| | | | TNFRSF4, APOA1, IRF7, |
| | | | SOX2 |
| | GO:0034340 | Response to type 1 | ISG15, IFIT1, IFIT2, IFIT3, |
| | | interferon | MX1, PTPN6, IRF7 |
| | GO:0045069 | Regulation of viral | ISG15, IFIT1, MX1, CCL5, |
| | | genome replication | CXCL8, TNF |
| | GO:0045071 | Negative regulation of | ISG15, IFIT1, MX1, CCL5, TNF |
| | | viral genome | |
| | | replication | |
| | GO:0071345 | Cellular response to | ISG15, IFIT1, IFIT2, IFIT3, |
| | | cytokine stimulus | MX1, CCL5, CCL2, CXCL8, |
| | | | TNF, TRAF1, PTPN6, |
| | | | TNFRSF4, APOA1, IRF7 |
| rVSV- | GO:0009615 | Response to virus | CCL5, ISG15, IFIT1, IFIT2, |
| GFP, 6 hpi | | | IFIT3, HERC5 |
| | GO:0019079 | Viral genome | CCL2, CCL5, ISG15, IFIT1 |
| | | replication | |
| | GO:0034340 | Response to type 1 | ISG15, IFIT1, IFIT2, IFIT3 |
| | | interferon | |
| | GO:0045071 | Negative regulation of | CCL5, ISG15, IFIT1 |
| | | viral genome | |
| | | replication | |
| | GO:0051607 | Defense response to | ISG15, IFIT1, IFIT2, IFIT3, |
| | | virus | HERC5 |
| | GO:0071345 | Cellular response to | CCL2, CCL5, ISG15, IFIT1, |
| | | cytokine stimulus | IFIT2, IFIT3, TRAF1, SFRP1, |
| | | | PTGS2 |

In all three cases (IVA 4hpi, IVA24hpi, and rVSV 6hpi), in any of the pathways identified (including defense response, viral life cycle, response to cytokine, interferons, negative regulation of viral genome regulation among others), ISG15 play a central role, thus emerging as an attractive candidate for knockout via CRISPR/Cas9.

### Example 3—Comparison of ISG15 Protein Sequences Across Species

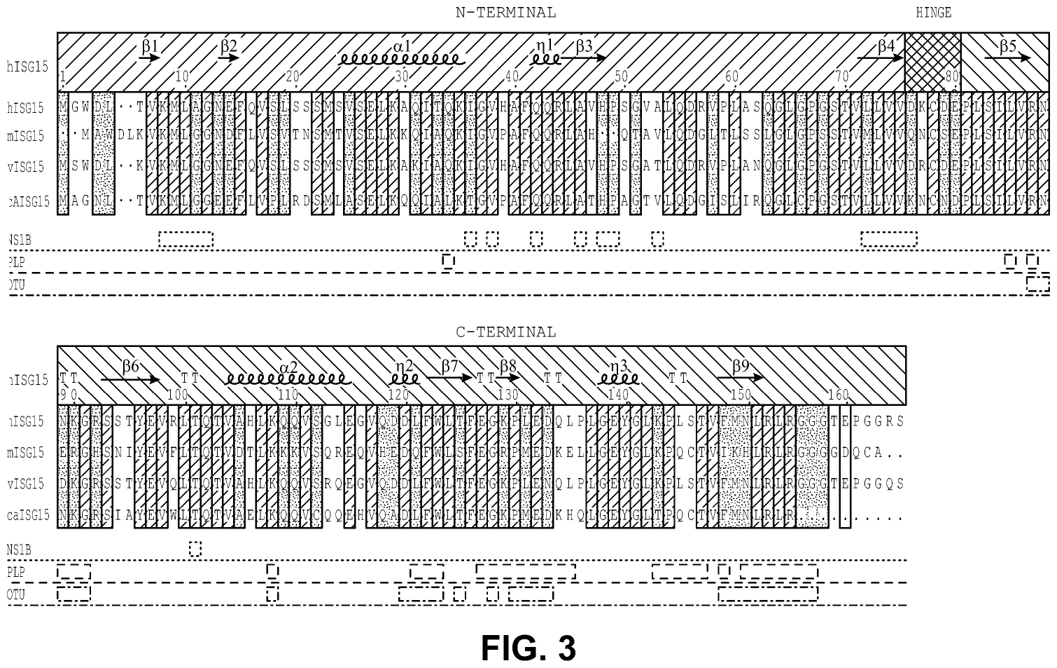

To verify that gene editing of ISG15 would lead to phenotypic modifications with regards to viral infection, the ISG15 protein sequences were compared across species of interest (i.e., from which cell lines used in vaccine production are derived), including human, mouse, Vero, and canine. ISG15 protein sequences were retrieved from RefSeq for Vero cells (XP_007979280.1), human (NP_005092.1), mice (NP_056598.2) and canine (XP_003639101.1) and are described herein as SEQ ID NO: 14 (Vero); SEQ ID NO: 15 (human); SEQ ID NO: 16 (mouse); and SEQ ID NO: 17 (canine). The sequences were aligned using T-Coffee and exported to the ESPript server for sequence alignment graphic design. See FIG. 3. Regions known to interact with viruses were also highlighted.

As shown in FIG. 3, mutations between human ISG15 and mouse ISG15 were similar to those between human ISG15 and Vero ISG15, especially at position 89, which was previously highlighted as a key player in the ability of Old World Monkey ISG15 (including Vero cells) to more efficiently ISGylate proteins compared to human ISG15, thus, giving some indications concerning the desired effects of ISG15 deletion in Vero cells. Pattyn E. et al., HyperISGylation of Old World monkey ISG15 in human cells, PLOS One 2008, 3 (6): e2427. In FIG. 3, the residues of ISG15 known to interact with the influenza NS1 protein, coronavirus PLPs, and nairovirus OTUs are also indicated beneath the sequence alignments. Dzimianski, J. et al., ISG15: it's Complicated, J. Mol Biol. 2019, 431 (21): 4203-4216.

### Example 4—Genomic Deletion of ISG15 Using CRISPR/Cas9 and Deletion Validation at the Genomic/Proteomic Level

The strategy used for the genomic deletion protocol relied on cellular delivery of a pair of chimeric single guide RNAs (sgRNAs) to create two double strand breaks (DSBs) at a locus in order to delete the intervening DNA segment by non-homologous end joining (NHEJ) repair. This method has been used to delete genes with a length between 1 to 10 kb (Bauer, D. E. et al., Generation of genomic deletions in mammalian cell lines via CRISPR/Cas9, J Vis Exp. 2015, 95: e52118) and was applied here for the deletion of the ISG15 gene's CDS regions. Genomic deletions may in certain instances be advantageous to homology-directed repair (HDR) or single-site small indel production. The high frequency of deletions limits the number of clones needed to be screened to find clones of interest, and monoallelic and biallelic deletions can be easily identified via PCR, thus avoiding more labor-intensive methods. Additionally, given that a significant portion of the gene of interest is deleted, reliable loss-of-function alleles can be obtained.

A pair of guide RNAs was designed using the freely available online tools CRISPOR and EuPaGDT, which already included the Vero cell genome in their list of custom genomes. These tools helped identify guide sequences that minimize identical genomic matches or near-matches to reduce the risk of cleavage away from target sites (off-target effects). The guide sequences contained a 20-mer (“protospacer sequence”) upstream of an “NGG” sequence (“protospacer adjacent motif” or PAM) at the genomic recognition site. The plasmid structures pX458 (Addgene plasmid ID 48138), purchased from GenScript, contained GFP as a selectable marker and one of the two designed gRNAs guide or B, wherein guide A was A guide ACCAGCATTCGAGCAAGATCAAGG (SEQ ID NO: 33) and guide B was GGAAACCGAAACTTGGCCACCGG (SEQ ID NO: 34).

Delivery of the CRISPR/Cas9 plasmids was done by electroporation. Four vials, each containing 2.6×10⁶ cells in 90 μL of growth medium, were prepared for transfection. The cells were washed two times in ice-cold phosphate-buffered saline (PBS), resuspended, and transferred to a 4 mm gapped cuvette. Four tubes of 10 mL growth media were prepared and put into the incubator for 10 minutes. 5 μg of each CRISPR/Cas9 construct containing guide A and guide B were mixed with the Vero cell suspensions, and the samples were immediately pulsed using an electroporator at 250 volts square wave for 20 ms. The cells were then diluted into the previously-prepared 10 mL prewarmed complete growth media and plated in a T75 cm² flask before incubation at 37° C., 5% CO₂ for 48 hours. For all studies, untransfected cells were included as a negative control.

Following CRISPR/Cas9-based genomic deletion of ISG15 CDS region, several validation steps were designed to confirm plasmid delivery using a GFP reporter and cell sorting, intended deletion using PCR, protein deletion using western blot, and deletion phenotypic effects via viral infection and virus production quantification.

The top ˜3% of GFP positive cells were sorted using flourescence activated cell sorting (FACS) in order to enrich for cells that received high levels of the CRISPR/Cas9 constructs. The sorted cells were individually plated into 96-well plates containing 100 μl per well of cell culture media using FACS sorter. The clones were incubated at 37° C. for 3 weeks. The resulting monoclonal colonies were passaged and split to proceed with validation steps.

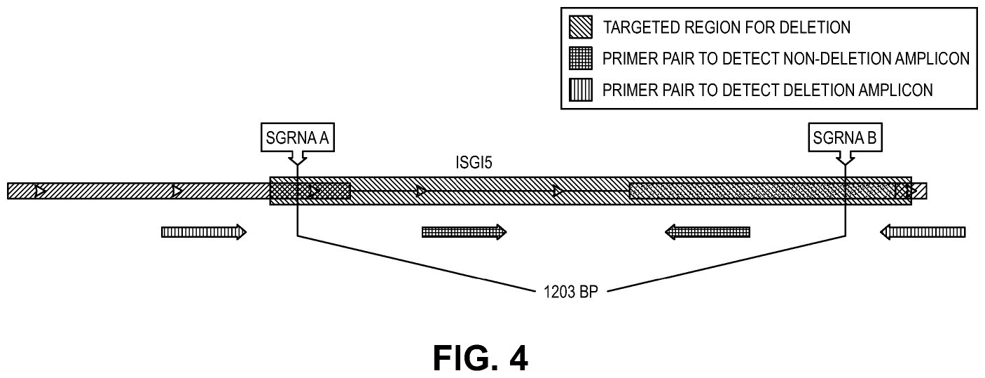

PCR was used to validate the intended genomic deletion of ISG15 CDS region. To confirm genomic deletion, two pairs of PCR primers were designed, as shown in FIG. 4. The first primer, sgRNA A, flanked inside the deletion region (non-deletion band), and the second primer, sg RNA B, flanked outside the deletion region (deletion band), thus allowing screening for deletion bands and non-deletion bands. The sgRNA A had a forward primer of GTCCCAGCTCTGCAGACATTA (SEQ ID NO: 35) and a reverse primer of GAGCTCGGCCAGGTTCTAAG (SEQ ID NO: 36). The sgRNA B had a forward primer of CCTCGAGGCTGTAACTGCAA (SEQ ID NO: 37) and a reverse primer of ACCATAGGGGTGTTTTCCGT (SEQ ID NO: 38).

In the absence of deletion, the deletion band is often too large to efficiently amplify. Primers at least 100 bp from the predicted cleavage site were used to ensure detection would not be impacted by a small indel at the sgRNA target site. The genomic DNA was extracted from each clone using Invitrogen PureLink Genomic DNA Mini Kit, and the DNA concentration was measured. Each clone was screened for both non-deletion band and deletion band detection using the following PCR protocol: for each detection, a 25 μL PCR reaction containing 12.5 μL master mix, 0.5 μL forward primer (10 μM), 0.5 μL reverse primer (10 μM), 100 ng gDNA, and H₂O up to 25 μL was run in the thermocycler (98° C. for 30s, 35 cycles of (98° C. for 10 sec, 60° C. for 30s, 72° C. for 1 min), and 72° C. for 2 min). The PCR products were then run on 2% agarose gel at 10 V/cm using 1× Tris-acetate-EDTA (TAE) buffer. The samples were examined for the detection of non-deletion and deletion bands using a Chemidoc (Biorad) and clones with biallelic deletions were passaged and split for cell banking and further validation analysis. This validation was repeated after a week for quality control.

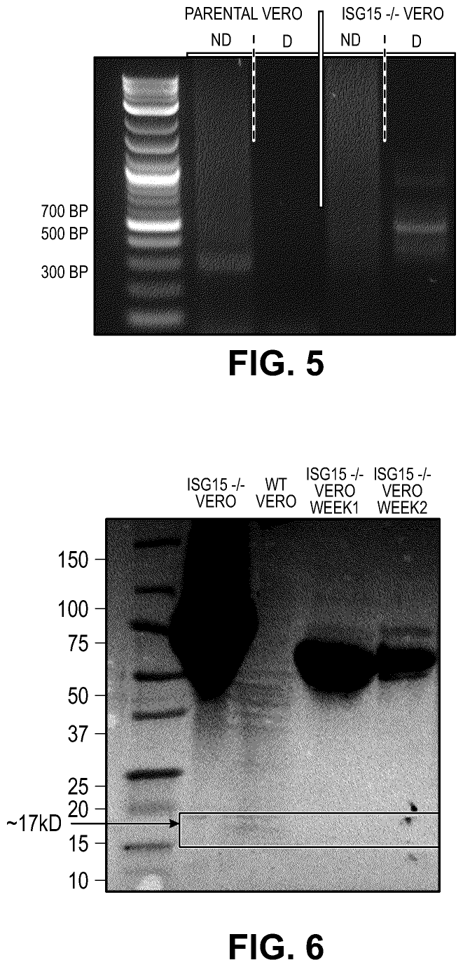

As shown in FIG. 5, biallelic clones are demonstrated by the absence of a non-deletion band and the presence of a deletion band for the ISG15 CDS region. Among 100 clones screened, 6 were identified with a biallelic deletion and good fitness (via monitoring of the clones doubling time).

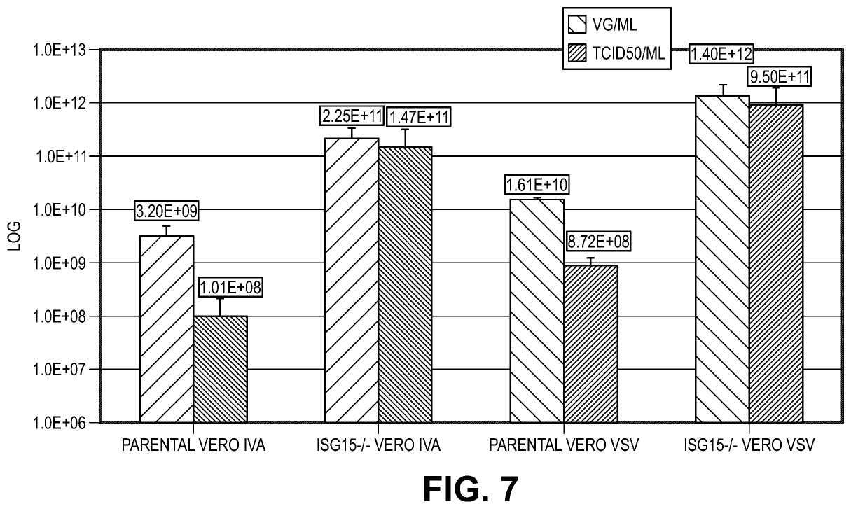

At the protein level, Western Blot analysis was performed to further confirm ISG15 deletion. In this protocol, 20 μL of each cell lysate sample was mixed with SDS loading buffer, separated on SDS-PAGE gels (BioRad Criterion TGX Precast gels), and transferred to polyvinylidene difluoride (PVDF) membranes. Immunoblotting was performed using the relevant antibodies (anti-ISG15, Invitrogen). Horseradish peroxidase coupled secondary antibodies were detected with the BioRad Clarity Western ECL substrate. The resulting signals were imaged with a Chemidoc (BioRad) and analysed by ImageJ. The Western Blot analysis showed that none of the previously selected clones had a band at 15-17 kDa that was visible in parental or wild-type Vero cells. See FIG. 6, showing the absence of a ISG15 band in ISG−/− cells, while the band at about 17 kDa is present in the parental Vero cells.

### Example 5—ISG−/− Vero Cell Virus Production Quantification

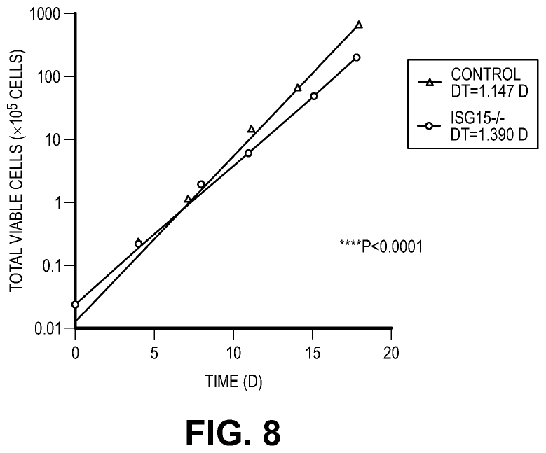

To quantify the viral production of IVA and rVSV-GFP in parental and ISG−/− Vero cells, triplicates of the cells were cultured and infected at MOI 10 with either IVA Puerto Rico 8 or rVSV-GFP. The supernatant for each sample was harvested 24 hours post infection and virus production was quantified via ddPCR (viral genome) and TCID50 (infectious viral particles). Infection of the engineered clones showed significant increases in both total viral particle production and in infectious viral particles. Indeed, an increase of 70.3-fold of total viral particles was observed for IVA infection, and an increase of 87-fold was shown for rVSV-GFP. Interestingly, the ratio of infectious viral particles/total viral particles also significantly increased, going from 0.0316 to 0.653 for IVA and from 0.0542 to 0.679 for rVSV-GFP. The results are shown in Table 5 below and illustrated graphically in FIG. 7, wherein STD is calculated as the standard deviation across the population.

TABLE 5
Production of IVA and rVSV-GFP viral particles

| | Average | | | |
|---|---|---|---|---|
| | viral | | | |
| | genome | Vg/mL | Average | TCID₅₀ |
| Sample | (Vg)/mL | STD | TCID₅₀ | STD |
| Parental Vero | 3.20 × 10⁹ | 1.82 × 10⁹ | 1.01 × 10⁸ | 1.13 × 10⁸ |
| IVA | | | | |
| ISG-/-Vero | 2.25 × 10¹¹ | 1.14 × 10¹¹ | 1.47 × 10¹¹ | 1.83 × 10¹¹ |
| IVA | | | | |
| Parental Vero | 1.61 × 10¹⁰ | 1.06 × 10⁸ | 8.72 × 10⁸ | 4.13 × 10⁸ |
| rVSV-GFP | | | | |
| ISG-/-Vero | 1.40 × 10¹² | 8.26 × 10¹¹ | 9.50 × 10¹¹ | 9.50 × 10¹¹ |
| rVSV-GFP | | | | |

These results demonstrate that deletion of the CDS region of ISG15 from Vero cells can increase viral particle production and infectious viral particle production of IVA and rVSV.

### Example 6—Growth Kinetics of ISG15−/− and Control Cell Lines

A pharmaceutical industrialized process for vaccine manufacturing in large-scale bioreactors was initiated from thawing a frozen vial of master cell bank seed into cell culture treated flasks or cell factories such as polystyrene Corning® CellSTACK® chambers (Corning).

Initially a quality control experiment was run in which ISG15−/− and control cell lines were cultured in serum free, ultra-low protein media containing no proteins, peptides, or other components of animal or human origin (VP-SFM AGT™ media (Thermo Fisher Scientific), using pharmaceutical grade reagents and equipment. Cells were propagated every 3 to 4 days at a seeding density of 0.44×10⁵ cells/cm² or 0.24×10⁵ cells/cm², respectively. The cells were grown in static culture at 37° C. and 5% CO₂ in a humidified incubator (Sanyo). Cells were passaged twice weekly using trypsin (Roche) as a dissociation reagent, which was inhibited by trypsin inhibitor (Sigma) in sodium citrate buffer. The passage process for ISG15−/− involved optimization from standard industrial procedures. This optimization included the addition of a wash step with sodium citrate buffer at 37° C. prior to trypsinization and conducting trypsinization at 37° C. in 5% CO₂ in a humidified incubator. However, the duration of trypsin exposure and concentration of trypsin were unmodified and remained consistent with cell culture of the control cell line.

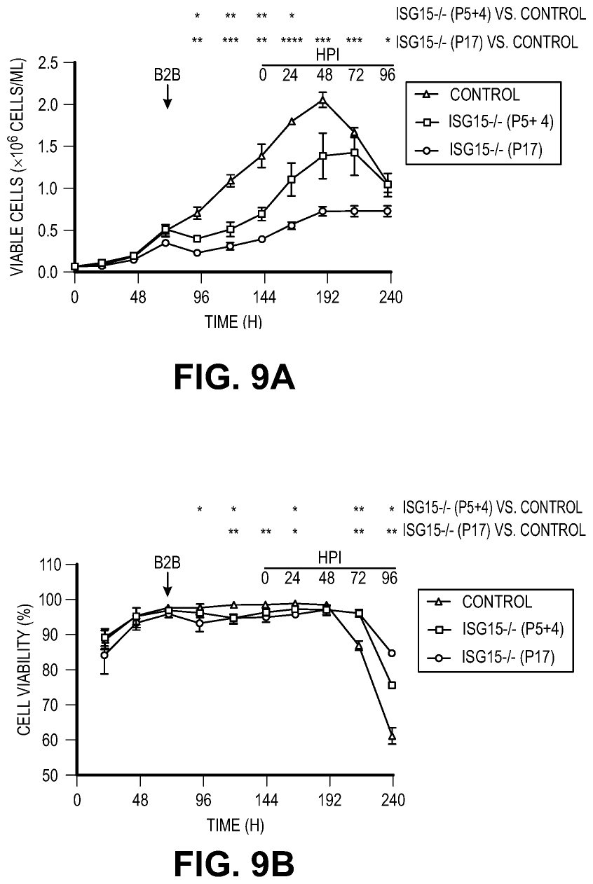

As shown in FIG. 8, the growth rate of ISG15−/− cells was significantly lower than the control cell line. FIG. 8 shows that the ISG15−/− cell line had a doubling time of 1.390 days compared to the control cell line doubling time of 1.147 days (P<0.0001). The growth rate (k) of ISG15−/− and the control cell line were 0.4987 and 0.6045 days, respectively. The goodness of fit for the nonlinear regression of ISG15−/− and the control cell line were 0.9997 and 1.000, respectively. However, it was confirmed that both the ISG15−/− and control cell lines can be sufficiently expanded in static culture to generate material to seed bioreactors, as the yield differed by only 2-fold within two weeks of culture.

### Example 7—Kinetic Analysis of Cell Performance in Bioreactors

A pharmaceutical industrial process for adherent Vero cells in vaccine manufacturing employs the use of microcarriers to propagate adherent cells in bioreactors. A quality control experiment was conducted to characterize the propagation of ISG15−/− cells using microcarriers in bioreactors.

Cells from two different passages of ISG15−/− cells lines (p5+4 and p17, as indicated above), along with control cells were collected from cell culture treated flasks and seeded on Cytodex® 1 microcarriers at a final density of 1 g/L (Cytiva) and a seeding density of 20,000 cells/cm². Cells were cultured in bioreactors at 37° C., with dissolved oxygen at 30%, and a pH of 7.2. The pH was regulated using sparged CO₂ and sodium bicarbonate addition, in a 12-way single-use bioreactor system (AMBR250, Sartorius Stedim), in 0.2 L of (VP-SFM AGT™ media (Thermo Fisher Scientific), using pharmaceutical grade reagents and equipment.

The total cells were expanded within the same bioreactor. 72 hours after seeding the bioreactors, cells were dissociated from microcarriers using 12.5 U/mL trypsin (Roche) and mechanical agitation using the bioreactor impellers for 10 minutes. The dissociation process was inhibited using trypsin inhibitor (Sigma) in sodium citrate buffer. Cell solutions were visually inspected to confirm cell dissociation from microcarriers. The total Cytodex® 1 microcarrier (Cytiva) concentration was increased to 4 g/L, and the culture continued for an additional 72 hours.

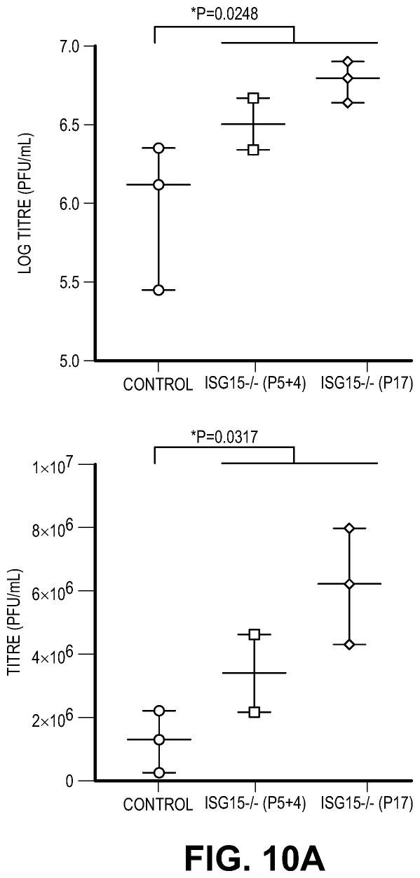

Media exchanges were conducted 24 hours after each cell passage. Media exchanges were performed by temporarily pausing impeller agitation, such that cells adherent to microcarriers settled by gravity, before removing 80% of the total bioreactor volume from the surface of the solution in the bioreactor. Three and six independent bioreactors were tested per control cell line and ISG15−/− cell line (3 at each passage indicated), respectively. Consistent with static cell culture in flasks, the total cell count from bioreactors containing ISG15−/− cells was lower than bioreactors containing control cells. As shown in FIGS. 9A and 9B, a two-way ANOVA, Dunnett's multiple comparisons test showed that total cell count and cell viability was significantly lower for the ISG15−/− cells than for the control cells.

### Example 8—Kinetic Analysis of Virus Production in Bioreactors

Upon characterization of the propagation of ISG15−/− cells using microcarriers in bioreactors as discussed in Example 7 above, all bioreactors were infected with RSV at a multiplicity of infection (MOI) of 0.01, which is representative of a pharmaceutical industrialized bioprocess.

The RSV infection was performed by temporarily pausing impeller agitation, such that cells adherent to microcarriers settled by gravity, before removing 80% of the total bioreactor volume from the surface of the solution in the bioreactor. Fresh media supplemented with 0.1% (volume/volume) SyntheChol® (Sigma Aldrich) was added to a final volume of 0.2 L. The virus stock was then added at an MOI of 0.01, adjusted to the number of total live cells of each independent bioreactor, and then impeller agitation was resumed. During the infection period, the bioreactor temperature reduced to 34° C., and the pH increased to 7.3. Cultures were maintained for 4 days following infection, with daily sample analysis including total and live cell counts. Infectious titre was tested by the plaque assay 72 hours post-infection. As shown in FIGS. 9A and 9B, control cells were significantly less viable than ISG15−/− cells at 72 hours post-infection, which is the standard time point for crude harvest following viral production.

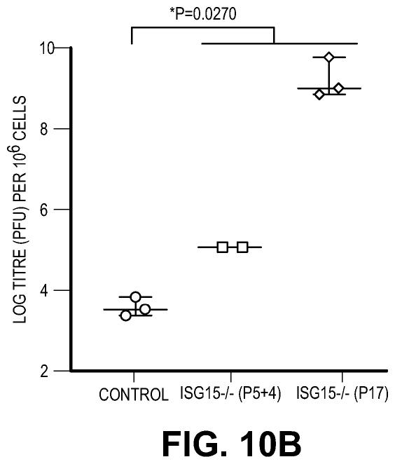

Samples were tested for log infectious viral titre using the plaque assay in parallel from samples that were cryopreserved in 1×HSG and stored at −80° C. until testing. The results are shown in FIG. 10A based on unpaired two-tailed T-tests. As shown in FIG. 10A, the log titre (PFU/mL) was significantly higher from bioreactors using ISG15−/− cells compared to control lines cells after 72 hours. At 72 hours post-infection, the increase in viral particle production was a 0.6967 log difference (P=0.0248) of infectious titre per liter of culture in crude harvest material for vaccine manufacturing. These results demonstrate that deletion of the CDS region of ISG15 from Vero cells can increase viral particle production and infectious viral particle production of RSV using pharmaceutical industrialized process in bioreactors using microcarriers. The increase in viral particle production was about a 300% difference of infectious titre per million cells in crude harvest material for vaccine manufacturing.

Due to the significant difference of total cell density during infection and viral production, the total viral production per one million cells was determined, using unpaired, two-tailed T-tests as shown in FIG. 10B. Factored by an average difference of 32% less total cells in bioreactors containing ISG15−/−, as compared to bioreactors containing the control cell line during viral production, this viral productivity translates to an increase in viral particle production that was about a 1.5 log difference of infectious titre per million cells in crude harvest material for vaccine manufacturing. See FIG. 10B. The increase in viral particle production was about a 1000% difference of infectious titre per million cells in crude harvest material for vaccine manufacturing.

It is also noted that, as used in this disclosure and the appended claims, the singular forms “a”, “an”, and “the” include plural referents unless the context clearly dictates otherwise. Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination). Ranges may be expressed herein as from about one particular value, and/or to about another particular value. When such a range is expressed, another aspect includes from the one particular value and/or to the other particular value. Similarly, when values are expressed as approximations, by use of the antecedent about, it will be understood that the particular value forms another aspect. It will be further understood that the endpoints of each of the ranges are significant both in relation to the other endpoint, and independently of the other endpoint. All references cited in this disclosure are hereby incorporated herein in their entirety.

SEQUENCES
Chlorocebus sabaeus APOA1:
(SEQ ID NO: 1)
MKATVLTLAMLFLTGSQARHFWQEDEPPQTPWDRVKDLVTVYVEALKDSGKD
YVSQFEGSALGKQLNLKLLDNWDSVTSTVSKLREQLGPVTQEFWDNLEKETEGL
RQEMSKDLEEVKAKVQPYLDDFQKKWQEEMELYRQKVEPLRAELHDGARQKL
HELHEKLSPLGEEVRDRARAHVDALRTHLAPYSDELRQRLAARLEALKENGGAR
LAEYHAKASEHLSTLSEKAKPALEDLRQGLLPVLESFKVSFLSALEEYSKKLSTQ
Chlorocebus sabaeus CCL2:
(SEQ ID NO: 2)
MKVSAALLCLLLIAATFSPQGLAQPDAINAPVTCCYNFTNRKISVQRLASYRRITS
SKCPKEAVIFKTIVAKEICADPKQKWVQDSMDHLDKQIQTPKP
Chlorocebus sabaeus CCL5:
(SEQ ID NO: 3)
MKVSVAALAVILVATALCAPASASPYASDTTPCCFAYIARPLPRAHIKEYFYTSG
KCSNPAVVFVTRKNRQVCANPEKKWVREYINSLEMS
Chlorocebus sabaeus CYP19A1:
(SEQ ID NO: 4)
MVLEMLNPMHYNITSMVPEAMPAATMPILLLTGLFLLVWNYEGTSSIPGPGYCM
GIGPLISHGRFLWMGIGSACNYYNRVYGEFMRVWISGEETLIISKSSSMFHIMKHN
HYSSRFGSKLGLQCIGMHEKGIIFNNNPDLWKTTRPFFMKALSGPGLVRMVTVC
AESLKTHLDRLEEVTNESGYVDVLTLLRRVMLDTSNMLFLRIPLDESAIVVKIQG
YFDAWQALLIKPDIFFKISWLYKKYEKSVKDLKDAIEVLIAEKRRRISTEEKLEEC
MDFATELILAEKRGDLTRENVNQCILEMLIAAPDTMSVSLFFMLFLIAKHPNVEE
AIMKEIQTVVGERDVKIDDMQKLKVMENFIYESMRYQPVVDLVMRKALEDDVI
DGYSVKKGTNIILNIGRMHRLEFFPKPNEFTLENFAKNVPYRYFQPFGFGPRGCA
GKYIAMVMMKAILVTLLRRFHVKTLQGQCVERIQKIHDLSSHPDETKNMLEMIF
TPRNSDRCLEH
Chlorocebus sabaeus CXCL8:
(SEQ ID NO: 5)
MTSKLAVALLAAFLLSAALCEGAVLPRSAKELRCQCIKTYSKPIHPKFIKELRVIE
SGPHCVNTEIIVKLSDGRELCLDPKVPWVSRVVEKFLKRAESQNS
Chlorocebus sabaeus ELF3:
(SEQ ID NO: 6)
MAATCEISNIFSNYVSAMYSSEDSTLAPVPPAAAFGADDLVLTLSNPQMSLEGTE
KTSWSGEQPQFWSKTQVLDWISYQVEKNKYDASAIDFSRCDMDGATLCNCALE
ELRLVFGPLGDQLHAQLRDLTSSSSDELSWIIELLEKDGMAFQEALDPGPFDQGSP
FAQELLDDSQQASPYHPGSCGAGAPSPGSSDVSTAGTGASRSSHSSDSGGSDVDL
DPTDGKLFPRDGFPDCKKGDPKHGKRKRGRPRKLSKEYWDCLEGKKSKHELNE
GLMKWENRHEGVFKFLRSEAVAQLWGQKKKNSNMTYEKLSRAMRYYYKREIL
ERVDGRRLVYKFGKNSSGWKEEEVLQSRN
Chlorocebus sabaeus FOS:
(SEQ ID NO: 7)
MMFSGFNADYEASSSRCSSASPAGDSLSYYHSPADSFSSMGSPVNAQDFCTDLA
VSSANFIPTVTAISTSPDLQWLVQPALVSSVAPSQTRAPHPFGVPTPSAGAYSRAG
IVKTMTGSRAQSTGRRGKVEQLSPEEEEKRRIRRERNKMAAAKCRNRRRELTDT
LQAETDQLEDEKSALQTEIANLLKEKEKLEFILAAHRPACKIPDDLGFPEEMSVAS
LDLSGGLPEAATPESEEAFTLPLLNDPEPKPSVEPVKSISSMELKAEPFDDFLFPAS
SRPSGSETARSVPDMDLSGSFYAADWEPLHSGSLGMGPMATELEPLCTPVVTCTP
SCTAYTSSFVFTYPEADSFPSCAAAHRKGSSSNEPSSDSLSSPTLLAL
Chlorocebus sabaeus HERC3:
(SEQ ID NO: 8)
MLCWGYWSLGQPGISTNLQGIVAEPQVCGFISDRSVKEVACGGNHSVFLLEDGE
VYTCGLNTKGQLGHEREGNKPEQIGALADQHIIHVACGESHSLALSDRGQLFSW
GAGSDGQLGLMTTEDSVAVPRLIQKLNQQTILQVSCGNWHCLALAADGQFFTW
GKNSHGQLGLGKEFPSQASPQRVRSLEGIPLAQVAAGGAHSFALSLSGAVFGWG
MNNAGQLGLSDEKDRESPCHVKLLRTQKVVYISCGEEHTAVLTKSGGVFTFGAG
SCGQLGHDSMNDEVNPRRVLELMGSEVTQIACGRQHTLAFVPSSGLIYAFGCGA
RGQLGTGHTCNVKCPSPVKGYWAAHSGQLSARADRFKYHIVKQIFSGGDQTFVL
CSKYENSSPAVDFRTMNQAHYTSLINDETIAVWRQKLSEHNNANTINGVVQILSS
AACWNGSFLEKKIDEHFKTSPKIPGIDLNSTRVLFEKLMNSQHSMILEQILNSFESC
LIPQLSSSPPDVEAMRIYLILPEFPLLQDSKYYITLTIPLAMAILRLDTNPSKVLDN
WWSQVCPKYFMKLVNLYKGAVLYLLRGRKTFLIPVLFNNYITAALKLLEKLYK
VNLKVKHVEYDTFYIPEISSLVDIQEDYLMWFLHQAGMKARPSIIQDTVTLCSYP
FIFDAQAKTKMLQTDAELQMQVAVNGANLQNVFMLLTLEPLLARSPFLVLHVR
RNNLVGDALRELSIHSDIDLKKPLKVIFDGEEAVDAGGVTKEFFLLLLKELLNPIY
GMFTYYQDSNLLWFSDTCFVEHNWFHLIGITCGLAIYNSTVVDLHFPLALYKKLL
NVKPGLEDLKELSPTEGRSLQELLDYPGEDVEETFCLNFTICRESYGVIEQKKLIP
GGDNVTVCKDNRFTVYISTCERLNIPTDSGQAIYKNGAETHSL
Chlorocebus sabaeus HERC5:
(SEQ ID NO: 9)
MERRSRRKSRRNGRSTAGQTAASQPAKSPDAQLWLFPSAAGFYRALLRRAEVTR
QICCSPRRLAVLERGGAGVQVHQVLAGSGGARTPKCIKLGKNMKIHSMDQGAD
HMLILSSDGKPFEYDYSMKHLRSESILQEKKIIQITCGDYHSLALSKGGELFAWGQ
NLHGQLGVGRKFPSTTTPQIVEHLAGVPLAQISAGEAHSMALSMSGNIYSWGKN
EFGQLGLGHTESKDSPSLIEALDNQKVEFLACGGSHSALLTQDGLLFTFGAGKHG
QLGHNSTQNELRPCLVAELVGNRVTQIACGRWHTLAYVSDLGKVFSFGSGKDG
QLGNGGTLDQLIPLPVKVSSSEELKLESHTSEKELIMIAGGNQSILLWIKKENSYV
NLKRTIPTLNEGTVKRWIADVETKRWHSTKREIQEIFSSPACLTGSFLRKRRTTEM
MPVYLDLNKARNIFKELTQKDRITNMITTCLKDNLLKRLPFHSPHQEALEVFFLLP
ECPVMHLSNNWESLVVPFAKVVCKMSDPSSLVLEEYWATLQESTFSKLVQMFK
TAVVCQLDYWDESAEENGNVQALLEMLKKLHRVNQMKCQLPESIFQVDELLYR
LNFFVEVCRRCLWKMTVDTSENAGCWVIFSHFPFIFNHLSKIKLLHTDTLLKIEGK
KHKAYLMSAAIEEERESEFALMPTFGLTVRRNHLIEDVLNQLSQFENEDLRKELW
VSFSGEIGYDLVGVKREFFYCLFEEMIQPEYGMFMYPEGASCMWFPVRPKFEKK
RYFFFGLLCGLSLFNCNVANLPFPLALFKKLLDQMPSLEDLKELSPDLGKNLQTL
LDDEGDNFEEVFYIHFNVHWDRNDINLIPNGSSIIVNQTNKRDYVSKYIDYIFNDS
VKAVYEEFRRGFYKMCDEDIIKLFHPEELKDVIVGHTDYDWKTFEKNARYEPGY
NSSHPTIVMFWKAFHKLTLEEKKKFLVFLTGTDRLQTKDLKNMKITFCCPESWN
ERDPMRALTCFSVLFLPKYSTMEAVEEALQVAINNNRGFG
Chlorocebus sabaeus IFIT1:
(SEQ ID NO: 10)
MSTNGDNHQVKDSLEQLRCHFTWELFIEDDEMPDLENRVLDQIEFLDTKYNVGI
HNLLAYVKHLKGQNEEALKSLKEAEDLMQKEHANQASVRSLVTWSNFAWVYY
HMGRLAEAQAYLDKVENICKKPSNPFRYRMECPEIDCEEGWALLKCGGKNYER
AKACFEKALEGDHENPEFSTGYAISAYRLDGFKLATKGYRQFSLLPLRQAVSLNP
DNGYLKVLLALKLQDNGQEAEGEKYLEEALANMSSQTYVFRYAAKFYRRKGA
VDKALELLKKALQETPTSVLLHHQIGLCYKTQMIQLKEATKGQPRGQNREKIDK
MIRLAIFHFESAVEKKPTFEVAHLDLARMYIEAGNHRKAEETFQKLLCMKPVVEE
TMQDIHLQYARFQEFQKKSEINAIIHYLKAIKIEQASFIRDKSINSLKKLVLKKLQR
NALDLESLSLLGFVYKLKGNMNEALEYYERALRLAADFENSVRQGP
Chlorocebus sabaeus IFIT2:
(SEQ ID NO: 11)
MSENTKNSLESSLRQLKCHFTWNLIEGENSLDDFEDKVFYRTEFQNREFKATMC
NLLAYLKHLKGQNEEALECLCKAEELIQQEHADQAEIRSLVTWGNYAWVYYHM
GRLSDAQIYVDKVKHICEKFSSPYRIESPELDCEEGWTRLKCGGNQNERAKVCFE
KALEKKPKNPEFTSGLAIASYRLDNWPPSQNAIDPLRQAIQLNPDNQYLKVLLAL
KLHKMREEGEEEGEGEKLVQEALEKAPGITDVLRSAAKFYRRKDEPDKAIELLK
KALEYLPNNAYLHCQIGCCYRAKVLQVMNLRQNGIYGKRKLLELIGHAVAHLK
KADEANDNLFRVCSILASLHALADQYEEAEYYFQKEFSKELTPVAKQLLHLRYG
NFQLYQMKCEDKAIHHFIEGVKINQKSREKEKMKDKLQKIAKMRLSKNGADSE
ALHVLAFLQELNKKMQQADEDSERGLESGSLIPSASSWNGE
Chlorocebus sabaeus IFIT3:
(SEQ ID NO: 12)
MYNLLAYIKHLDGKNEAALECLRQAEELIQQEHADQAEIRSLVTWGNYAWVYY
HLGRLSDAQIYVDKVKQTCKKFSNPYSIEYPELDCEEGWTQLKCGRNERAKVCF
EKALEEKPNNPEFSSGLAIAMYHLDNNPEKQFSTDVLKQAIELSPDNQYVKVLLG
LKLQKMNKEAEGEQLVEEALEKAPCQTDVLRSAAKFYRRKGDLDKAIELFQRA
LESTPNNGYLYHQIGCCYKAKVRQMQNTGESEASGNKEKIEALKQYAMDYSNK
ALEKGLNPLDAYSDLAEFLEAECYQTPFSKEDPDAEKQQSHQHYYNLQKYNGKS
EDTALQRGLEGLSISKKSTEKEEIKDQPQNVSENLLPQNAPNYWYHQGLIHKQNG
DLLQAVKCYEKELGRLLRNAPSGIGSFFLSASELEDGSEEMGQDAVSSNPRELVS
NSE
Chlorocebus sabaeus IRF7:
(SEQ ID NO: 13)
MALAPERAAPRVLFGEWLLGEISSGCYEGLQWLDEARTCFRVPWKHFARKDLSE
ADARIFKAWAVARGRWPPSSRGGDPPPPEAEAAERASWKTNFRCALRSTRRFV
MLRDNSGDPADPHKVYALSPELGWREGPGTDQTEAEAPAAVRAPQGRPPGPFL
AHRDAGLQAPGPLPAPAGDKGDLLLQAVQQSCLADHLLTASWGADPVPAQAPG
EGQEGLPLTGACAGGPGLPAGELCTWAVEATPSPGSQPAALMTGEATAPEPLHQ
VEPYLAPSPSACTAVQEPSPGALDVTIMYKGRTVLQKVVGHPSCMFLYGPPDPA
VRATDPQQVAFPSPAELPDQKQLRYTEELLRHVAPGLQLELRGPQLWARRMGK
CKVYWEVGGPPGSASPSTPACLLPRNCDTPIFDFRVFFRELVEFRARQRRGSPCYT
IYLGFGQDLSARRPKEKSLVLVKLEPWLCRVHLEGTQREGVSSLDSSSLSLCLSST
NSLYDDIECLLMELEQPV
Chlorocebus sabaeus ISG15:
(SEQ ID NO: 14)
MSWDLKVKMLGGNEFQVSLSSSMSVSELKAKIAQKIGVHAFQQRLAVHPSGAT
LQDRVPLANQGLGPGSTVLLVVDRCDEPLSILVRNDKGRSSTYEVQLTQTVAHL
KQQVSRQEGVQDDLFWLTFEGKPLENQLPLGEYGLKPLSTVFMNLRLRGGGTEP
GGQS
Homo sapiens ISG15:
(SEQ ID NO: 15)
MGWDLTVKMLAGNEFQVSLSSSMSVSELKAQITQKIGVHAFQQRLAVHPSGVA
LQDRVPLASQGLGPGSTVLLVVDKCDEPLSILVRNNKGRSSTYEVRLTQTVAHL
KQQVSGLEGVQDDLFWLTFEGKPLEDQLPLGEYGLKPLSTVFMNLRLRGGGTEP
GGRS
Mus musculus ISG15:
(SEQ ID NO: 16)
MAWDLKVKMLGGNDFLVSVTNSMTVSELKKQIAQKIGVPAFQQRLAHQTAVL
QDGLTLSSLGLGPSSTVMLVVQNCSEPLSILVRNERGHSNIYEVFLTQTVDTLKK
KVSQREQVHEDQFWLSFEGRPMEDKELLGEYGLKPQCTVIKHLRLRGGGGDQC
A
Canine ISG15:
(SEQ ID NO: 17)
MLEPTAMAGNLTVKMLGGEEFLVPLRDSMLASELKQQIALKTGVPAFQQRLAT
HPAGTVLQDGISLIRQGLCPGSTVLLVVKNCNDPLSILVRNNKGRSIAYEVWLTQ
TVAELKQQVCQQEHVQADLFWLTFEGKPMEDKHQLGEYGLTPQCTVFMNLRLR
GGGGNWAGPGGQC
Chlorocebus sabaeus KRT15:
(SEQ ID NO: 18)
MTTTFLQTSSSTFGGGSTRGGSLLAGGGGFGGGSLYGGGGSRTISASSARFVSSGS
GGGYGAGMRVCGFGGGAGSAFGGGFGGGIGGGFGGGFGGGDGGLLSGNEKIT
MQNLNDRLASYLDKVRALEEANADLEVKIRDWYQKQAPTSPERDYSQYFKTTE
ELRDKIMATTIDNSRVILEIDNARLAADDFRLKYENELALRQSVEADINGLRRVL
DELTLAKTDLEMQIEGLNEELAYLKKNHEEEMKEFGSQLAGQVNVEMDAAPGV
DLTRVLAEMREQYEAMAEKNRRDAEAWFFSKTEELNKEVASNTEMIQTSKTEIT
DLRRTMQELEIELQSQLSMKAGLENSLAETECRYATQLQQIQGLIGGLEAQLSEL
RCEMEAQNQEYKMLLDIKTRLEQEIATYRSLLEGQDAKMADIGIREASSGGGGSS
SNFRINVEESVDGKVVSSRKREI
Chlorocebus sabaeus KRT19:
(SEQ ID NO: 19)
MTSYSYRQSSATSSFGGLGGGSVRFGPGVAFRAPSMHGGSGGRGVSVSSARFVS
SSSSGGYGGGYGGVLAASDGLLAGNEKLTMQNLNDRLASYLDKVRALEAANGE
LEVKIRDWYQKQGPGPSRDYSHYYTTIQDLRDKILGATIENSRIVLQIDNARLAA
DDFRTKFETEQALRMSVEADINGLRRVLDELTLARTDLEMQIEGLKEELAYLKK
NHEEEISVLRGQVGGQVSVEVDSAPGTDLAKILSDMRSQYEVMAEQNRKDAEA
WFTSRTEELNREVAGHTEQLQISRSEVTDLRRTLQGLEIELQSQLSMKAALEDTL
AETEARFGAQLAHIQALISGVEAQLGDVRADSERQNQEYQRLMDIKSRLEQEIAT
YRSLLEGQEDHYNNLSASKVL
Chlorocebus sabaeus MX1:
(SEQ ID NO: 20)
MVLSEVDIVKADPAAASQPLLLNGDADVAQKSPGSVAENNLCSQYEEKVRPCID
LIDSLRALGVEQDLALPAIAVIGDQSSGKSSVLEALSGVALPRGSGIVTRCPLVLK
LKKLVNEDEWRGKVSYQDYEIEILDASEVEKEINKAQNTIAGEGMGISHELITLEI
SSRDVPDLTLIDLPGITRVAVGNQPPDIGYKIKTLIRKYIQRQETINLVVVPSNVDI
ATTEALSMAQEVDPEGDRTIGILTKPDLVDKGTEDKVVDVVRNLVFHLKKGYMI
VKCRGQQEIQDQLSLSEALQREKIFFEDHPHFRDLLEEGKATIPCLAEKLTSELIAH
ICKSLPLLENQIKESHQGITEELQKYGVDIPEDENEKMFFLIDKINTFNQDITALIQG
EETVGEDDSRLFTRLRREFHKWGIIIENNLQEGHKITSRKMQKFENQYRGRELPG
FVNYRTFETIIKQQIKALEEPAVNMLHTVTDMVRLAFADVSMKNFEELFNLHRT
AKSKIEDIRTEQEREGEKLIRLHFQMEQIVYCQDQVYRGALQKVREKELEEEKKK
KSWDVGTFQPSSTDSSMEEIFQHLMAYHQEASKRISSHIPLIIQFFMLQTYGQQLQ
KAMLQLLQDKDTYSWLLKERSDTSDKRKFLKERLARLTQARRRLAQFPG
Chlorocebus sabaeus NGFR:
(SEQ ID NO: 21)
MRAGATGRAMDGPRLLLLLLLGVSLGGAKEACPTDLYTHSGECCKACNLGEGV
AQPCGANQTVCEPCLDSVTFSDVVSATEPCKPCTECVGLQSMSAPCVEADDAVC
RCAYGYYQDETTGRCEACRVCEAGSGLVFSCQDKQNTVCEECPDGTYSDEANH
VDPCLPCTVCEDTERQLRECTRWADAECEEIPGRWITRSTPPEGSDSTAPSTQEPE
APPEQDLIASTVADVVTTVMGSSQPVVTRGTTDNLIPVYCSILAAVVVGLVAYIA
FKRWNSCKQNKQGANSRPVNQTPPPEGEKLHSDSGISVDSQSLHDQQPHTQTAS
GQALKGDGGLYSSLPPAKREEVEKLLNGSAGDTWRHLAGELGYQPEHIDSFTHE
ACPVRALLASWATQDSATLDALLAALRRIQRADLVESLCSESTATSPV
Chlorocebus sabaeus PTGS2:
(SEQ ID NO: 22)
MLARALLLCAVLALGHAANPCCSYPCQNRGVCMSVGFDQYKCDCTRTGFYGE
NCSTPEFLTRIKLFLKPTPNTVHYILTHFKGFWNVVNNIPFLRNAIMSYVLTSRSH
LIDSPPTYNVDYGYKSWEAFSNLSYYTRALPPVPDDCPTPLGVKGKKQLPDSNEI
VEKFLLRRKFIPDPQGTNMMFAFFAQHFTHQFFKTDHKRGPAFTKGLGHGVDLN
HIYGETLDRQHKLRLFKDGKMKYQIIDGEMYPPTVKDTQAEMIYPPQVPEHLQF
AVGQEVFGLVPGLMMYATIWLREHNRVCDVLKQEHPEWGDEQLFQTSRLILIGE
TIKIVIEDYVQHLSGYHFKLKFDPELLFNKQFQYQNRIAAEFNTLYHWHPLLPDN
FQIHDQKYNYQQFIYNNSILLEHGITQFVESFTRQIAGRVAGGRNVPPAVQKVSQ
ASIDQSRQMKYQSFNEYRKRFMLKPYESFEELTGEKEMSAELEALYGDIDAVEL
YPALLVEKPRPDAIFGETMVEVGAPFSLKGLMGNVICSPAYWKPSTFGGEVGFQI
INTASIQSLICNNVKGCPFTSFSVPDPELIKTVTINASSSRSRLDDINPTVLLKERSTE
L
Chlorocebus sabaeus PTPN6:
(SEQ ID NO: 23)
MPSPRMVRWFHRDLSGLDAETLLKGRGVHGSFLARPSRKNQGDFSLSVRVGDQ
VTHIRIQNSGDFYDLYGGEKFATLAELVEYYTQQQGVLQDRDGTVIHLKYPLNC
SDPTSERWYHGHMSGGQAETLLQAKGEPWTFLVRESLSQPGDFVLSVLSDQPKA
GPGSPLRVTHIKVMCEGGRYTVGGSETFDSLTDLVEHFKKTGIEEASGAFVYLRQ
PYYATRVNAADIENRVLELNKKKESEDTAKAGFWEEFESLQKQEVKNLHQRLE
GQRPENKGKNRYKNILPFDHSRVILQGRDSNIPGSDYINANYIKNQLLGPDENTK
TYIASQGCLEATVNDFWQMAWQENTRVIVMTTREVEKGRNKCVPYWPEVGTQ
RVYGPYSVTNCGEHDTTEYKLRTLHVSPLDNGDLIREIWHYQYLSWPDHGVPSE
PGGVLSFLDQINQRQESLPHAGPIIVHCSAGIGRTGTIIVIDMLMENISTKGLDCDI
DIQKTIQMVRAQRSGMVQTEAQYKFIYVAIAQFIETTKKKLEVLQSQKGQESEYG
NITYPPAMKNAHAKASRTSSKHKEDVYENLHSKNKREEKVKKQRSADKEKSKG
SLKRK
Chlorocebus sabaeus RET:
(SEQ ID NO: 24)
MAKATSGAAGLRLLLLLLLLPLLGKVALGLYFSRDAYWEKLYVDQPAGTPLLY
VHALRDAPEEVPSFRLGQHLYGTYRTRLHENNWICIQEDTGLLYLNRSLDRSSW
EKLSGRNRGFPLLTVYLKVFLSPTFLREGECQWPGCARVYFSFFNTSFPACTSLKP
RELCFPETRPSFRIRENRPPGTFHQFRLLPVQFLCPNISVAYRLLEGEGLPFRCAPD
SLEVSTRWALDREQREKYELVAVCTVHAGAREEVVMVPFPVTVYDEDDSAPTF
PAGVDTASAVVEFKRKEDTVVATLRVFDADVVPASGELVRRYTSTLLPGDTWT
QQTFRVEHWPNETSVQANGSFVRATVHDYRLVLNRNLSISENRTMQLAVLVND
SDFQGPGAGVLLLHFNVSVLPVSLHLPSSYSLSVSRRARRFAQIGKVCVENCQAF
SGINVQYELHSSGANCSTLGVVTSAEDTSGILFVNDTKALRRPKCAELHYMVVA
TDHQTSRQAQAQLLVTVEGSYVAEEAGCPLSCAVSKRRPECEECGGLGSPTGRC
EWRQGDGKGITRNFSTCSPSTKTCPDGHCDVVETQDINICPQDCLRGSIVGGHEP
GEPRGIKAGYGTCNCFPEEEKCFCEPEDIQDPLCDELCRTVIAAAVLFSFIVSVLLS
AFCIHRYHKFAHKPPIPSAEMTFRRPAQAFPVSYSSSGTRRPSLDSMENQVSVDAF
KIPEDPKWEFPRKNLVLGKTLGEGEFGKVVKATAFRLKGRAGYTTVAVKMLKE
NASPSELRDLLSEFNLLKQVNHPHVIKLYGACSQDGPLLLIVEYAKYGSLRGFLR
ESRKVGPGYLGSGGSRNSSSLDHPDERALTMGDLISFAWQISRGMQYLAEMKLV
HRDLAARNILVAEGRKMKISDFGLSRDVYEEDSYVKRSKGRIPVKWMAIESLFD
HIYTTQSDVWSFGVLLWEIVTLGGNPYPGIPPERLFNLLKTGHRMERPDNCSEEM
YRLMLQCWKQEPDKRPVFADISKDLEKMMVKSRDYLDLAASTPSDSLLYDDGL
SEEETPLVDCNNAPLPRALPSTWIENKLYGMSDPNWPGESPVPLTRADGTNTGFP
RYANDSVYANWMLSPSAAKLMDTFDS
Chlorocebus sabaeus ROS1:
(SEQ ID NO: 25)
MKNIYCLILKLVNFATLGCLWISVVQCTVLNSCLKSCVTNLGQQLDLGTPHNLSE
PCIQGCHFWNSVDQKNCALKCNDTYATICERESCEVGCSSAEGAYEEEVLGNAD
LPTAPFASSIGSHDMTLRWKSANFSGVKYIIQWKYAQLLGSWTYTKTVSKLSYV
VEPLHPFTEYIFRVVWIFTAQLQLYSPPSPSYRTHPHGVPETAPLIRNIESSSPDTVE
VSWDPPQFPGGPILGYNLRLISKNQKLDAGTQRTSFQFYSTLPNTMYRFSTAAVN
EVGEGPEAESSITTSSSAVQEEEQWLFLSRKTSLRKRSLKHLVDEAHCLRLDAIYH
NITGISVDVHQQTVYFSEGTLIWVKKAVNMSDVSDLKIFYRGSGLISSISVDWLY
QRMYFIMEELVCVCDLENCSNIEEITPPSISAPRKIVADSYNGYVFYLLRDGIYRA
DLPVPSGRRAETVRIVESCTLKDFAIKPQSKRIVYFNDTAQVFMSTFLDGSASHLI
LPRIPFADVKSFACENNDFLVTDGKVIFQQDALSFNEFIVGCDLSHIEEFGFGNLVI
FGSSSQLHPLPGRPQELSVLFGSHQAFVQWKPPALAIGASPSAWQNWTYEVKVS
TQDPPEVTRIFSNISGTMLNVPELQSATKYKVSVRASSPKRPGPWTEPSVGTTLVP
ASEPPFIMAVKEDGLWSKPLNSFGPGEFLSSDIGNVSDMDWYNNSLYYSDTKGD
VFVWLLNGMDISENYHLPGIAGAGALAFEWLGHFLYWAGKTYVIQRQSVLTGH
TDIVTHVKLLVNDMVVDSVGGYLYWTTLYSVESTRLNGESSLILQAQPWFSGKK
VIALTLDLSDGLLYWLVQDSQCIHLYTAVLRGQSTGDTTITEFAAWSTSEISQNA
LMYYSGRLFWINGFRIITTQEIGQRTSVSVLEPAKFNQFTIIQTSLKPLPGNFSFTPK
VIPDSVQESSFRIEGNASSFQILWNGPPAVDWGVVFYSVEFSAHSKFLASERHSLP
VFTVEGLEPYALFNLSVTPYTYWGKGPKTALSLRAPETVPSAPENPRIFILPSGKC
CNKNEVVVEFRWNKPKHENGVLTKFEIFYKISNQSITNKTFEDWIAVDVTPSVMS
FQLEGMSPRCFVAFQVRAFTSKGPGSFADVVKSTTSEINPFPHLITLLGNEIAFLD
MDQNQVVWTFSAERVISAICYTADNELGYYAEGDSLFLLNLHNRSSSELFRDSLV
FDITVITIDWISRHLYFALKESQNGMQIFDVDLEHKVKHPREVKIHNRNSTIISFSV
YPLLSRLYWTEVSSFGYQMFYYSIINCTLHRILQPTATNQQNKRNQCSCNVTEFE
LSGAMAIDTSNLEKSLIYFAKTQEIWAMDLEGCHCWRVITVPAMLGKTLASLTV
DGDFIYWIIIAKDSTRIYQAKKGNGAIVSQVKALRSRHILAYSSVMQPFPGKAFLS
LASETVEPTILNATNTSLTIRLPLAKTTLTWYGITSPTPTYLVCYAEVNDRKNSSD
LKYRILEFQDSIALIEDLQPFSTYMIQIAVKNYYSDPLEHLPPGKEIWGKTKNGVP
EAVQLINTTVRSDTSLIISWKESHKPNGPQESVRYQLAISHLAPIPETPLRQSEFPN
GRLTLLVTGLSGGNIYVLKVLACHSEEMWCTESHPVTVEMENTPEKPYSLVPEN
TSLQFNWKAPLNVNLIRFWAELQKWKYNEFYHVKTSCNQGPAYVCNITNLQPH
TSYNVRVVVVYKTGENSTSLPESFKTKAGVPSKPGIPKLLEGSKNSIQWEKAEDN
GCRIIYYILEIRKSTSNLQNQNLRWKMTFNGSCSSICTWKSKNLKGIFQFRVVAAN
NLGFGEYSGISENTVLVGDDFWIPETSFILSIIVGIFLVITIPLTFVWLRRLKNQKSA
KEGLTVLINEDKELAELRGLAAGVGLANACYAIHTLPTQEEIENLPAFPREKLTLR
LLLGSGAFGEVYEGTAVDILGAGSGEIKVAVKTLKKGSTDQEKIEFLKEAHLMSK
FNHPNILKQLGVCLLNEPQYIILELMEGGDLLTYLRKARMATFYGPSLTLVDLVD
LCVDISKGCVYLEQMHFIHRDLAARNCLVSVKDYTSPRIVKIGDFGLARDIYKND
YYRKRGEGLLPVRWMAPESLMDGIFTTQSDVWSFGILIWEILTLGHQPYPAHSNL
DVLNYVQTGGRLEPPRNCPDDLWNLMTQCWAQEPDQRPTFHRIQDQLQLFRNF
FLNSIYQCRDEANTSGVINENFEGEDGDVICLNSDDIMPVALMETKNREGLNYM
VLATECGQGEEKSEGPLGSQESKSCGLRKEEKEPHADKDFCQEKQVAYCPPGKP
EGLNYACLSHTGYGDGAD
Chlorocebus sabaeus SFRP1:
(SEQ ID NO: 26)
MGSGRSAGGCRGAALEVLLALGAALLAVGLASEYDYVSFQSDIGPYQSGRFYTK
PPQCVDIPADLRLCHNVGYKKMVLPNLLEHETMAEVKQQASSWVPLLNKNCHA
GTQVFLCSLFAPVCLDRPIYPCRWLCEAVRDSCEPVMQFFGFYWPEMLKCDKFP
EGDVCIAMTPPNATEASKPQGTTVCPPCDNELKSEAIIEHLCASEFALRMKIKEVK
KENGDKKIVPKKKKPLKLGPIKKKDLKKLVLYLKNGADCPCHQLDNLSHHFLIM
GRKVKSQYLLTAIHKWDKKNKEFKNFMKKMKNHECPTFQSVFK
Chlorocebus sabaeus SOX2:
(SEQ ID NO: 27)
MYNMMETELKPPGPQQTSGGGGGGGNSTAAAAGGNQKNSPDRVKRPMNAFM
VWSRGQRRKMAQENPKMHNSEISKRLGAEWKLLSETEKRPFIDEAKRLRALHM
KEHPDYKYRPRRKTKTLMKKDKYTLPGGLLAPGGNSMASGVGVGAGLGAGVN
QRMDSYAHMNGWSNGSYSMMQDQLGYPQHPGLNAHGAAQMQPMHRYDVSA
LQYNSMTSSQTYMNGSPTYSMSYSQQGTPGMALGSMGSVVKSEASSSPPVVTSS
SHSRAPCQAGDLRDMISMYLPGAEVPEPAAPSRLHMSQHYQSGPVPGTAINGTLP
LSHM
Chlorocebus sabaeus SPP1:
(SEQ ID NO: 28)
MRIAVISFCLLGIAYALPVKQADSGSSEEKQLYNKYPDAVATWLKPDPSQKQNL
LAPQNAVSSEETNDFKQETLPSKSNESHDHMDDVDDEDDDDHVDSQDSIDSNDS
DEVDDTDDSHQSDESHQSDESDELVTDFPTDLPATEVFTPVVPTVDIYDGRGDSV
AYGLRSKSKKFRRPDIQYPDATDEDITSHVESEELNGAYKAIPVAQGLNVPSDWD
SRGKDSHETSQLDDHSAETHSHKHSRLYKRKASDDSNEHSDVIDSQERSKISREF
HSHEFHSHEDMLVVDPKSKEEDKHLKFRISHELDSASSEVN
Chlorocebus sabaeus TNF:
(SEQ ID NO: 29)
MSTESMIRDVELAEEALPRKTAGPQGSRRCWFLSLFSFLLVAGATTLFCLLHFGVI
GPQREEFPKDPSLFSPLAQAVRSSSRTPSDKPVAHVVANPQAEGQLQWLNRRAN
ALLANGVELTDNQLVVPSEGLYLIYSQVLFKGQGCPSNHVLLTHTISRIAVSYQT
KVNLLSAIKSPCQRETPEGAEAKPWYEPIYLGGVFQLEKGDRLSAEINLPDYLDF
AESGQVYFGIIAL
Chlorocebus sabaeus TNFRSF4:
(SEQ ID NO: 30)
MCVGARRLGRGPCAALLLLGLGLSTTAKLHCVGDTYPSNDRCCQECRPGNGMV
SRCNRSQNTVCRPCGPGFYNDVVSAKPCKACTWCNLRSGSERKQPCTATQDTV
CRCRAGTQPLDSYKPGVDCAPCPPGHFSPGDNQACKPWTNCTLAGKHTLQPASN
SSDAICEDRDPTPTQPQETQGPPARPTTVQPTEAWPRTSQRPSTRPVGVPRGPAVA
AILGLGLALGLLGPLAMLLALLLLRRDQRLPPDAPKAPGGGSFRTPIQEEQADAH
STLAKI
Chlorocebus sabaeus TRAF1:
(SEQ ID NO: 31)
MKQWKTRLGSGLESGPMALEQNLSDLQLQAAVEVAGDLEVDCYRAPCSESQEE
LALQHFMKEKLLAELEGKLRVFENIVAVLNKEVEASHLALATSIHQSQVDRERIL
SLEQRVVELQQTLAQKDQALGKLEQSLRLMEEASFDGTFLWKITNVTRRCHESA
CGRTVSLFSPAFYTAKYGYKLCLRLYLNGDGTGKRTHLSLFIVIMRGEYDALLP
WPFRNKVTFMLLDQNNREHAIDAFRPDLSSASFQRPQSETNVASGCPLFFPLNKL
QSPKHAYVKDDTMFLKCIVETST
Chlorocebus sabaeus VAV3:
(SEQ ID NO: 32)
MEPWKQCAQWLIHCKVLPANHRVTWDSAQVFDLAQTLRDGVLLCQLLNNLRA
HSINLKEINLRPQMSQFLCLKNIRTFLTACCETFGMRKSELFEAFDLFDVRDFGKV
IETLSRLSRTPIALATGIRPFPTEESINDEDVYKGLPDLIDETLVEDEEDLYDCVYG
EDEGGEVYEDLMKAEEAHQPKCPENDIRSCCLAEIKQTEEKYTETLESIEKYFMA
PLKRFLTAAEFDSVFMNIPELVKLHRNLMQEIHDSIVNKNDHNLYQVFINYKERL
VIYGQYCSGVESAISSLDYISKTKEDVKLKLEECSKRANNGKFTLRDLLVVPMQR
VLKYHLLLQELVKHTTDATEKANLKLALDAMKDLAQYVNEVKRDNETLREIKQ
FQLSIENLNQPVLLFGRPQGDGEIRITTLDKHTKQERHIFLFDLAVIVCKRKGDNY
EMKEIIDLQQYKIANNPTTDKENKKWSYGFYLIHTQGQNGLEFYCKTKDLKKKW
LEQFEMALSNIRPDYADSNFHDFKMHTFTRVTSCKVCQMLLRGTFYQGYLCFKC
GARAHKECLGRVDNCGRVNSGEQGTLKLPEKRTNGLRRTPKQVDPGLPKMQVI
RNYSGTPPPALHEGPPLHLQAGDTVELLRGDAHSLFWQGRNLASGEVGFFPSDA
VKPCPCVPKPVDYSCQPWYAGAMERLQAETELINRVNSTYLVRHRTKESGEYAI
SIKYNNEAKHIKILTRDGFFHIAENRKFKSLMELVEYYKHHSLKEGFRTLDTTLQF
PYKEPEHSTGQRVNRAGNSLLSPKVLGIAIARYDFCARDMRELSLLKGDVVKIYT
KMSANGWWRGEVNGRVGWFPSTYVEEDE
Guide A:
(SEQ ID NO: 33)
ACCAGCATTCGAGCAAGATCAAGG
Guide B:
(SEQ ID NO: 34)
GGAAACCGAAACTTGGCCACCGG
sgRNA A Forward Primer
(SEQ ID NO: 35)
GTCCCAGCTCTGCAGACATTA
sgRNA A Reverse Primer
(SEQ ID NO: 36)
GAGCTCGGCCAGGTTCTAAG
sgRNA B Forward Primer
(SEQ ID NO: 37)
CCTCGAGGCTGTAACTGCAA
sgRNA B Reverse Primer
(SEQ ID NO: 38)
ACCATAGGGGTGTTTTCCGT

1. An engineered cell line comprising a modification in an ISG15 gene,
wherein the modification in the ISG15 gene results in an increase in total viral particle production and/or infectious viral particle production as compared to a control cell line that is identical to the engineered cell line except for the modification in the ISG15 gene.

2. The engineered cell line of claim 1, wherein the increase in viral particle production is at least about 20%, such as at least about 40%, at least about 50%, at least about 60%, at least about 70%, at least about 80%, at least about 90%, at least about 100%, at least about 150%, at least about 200%, at least about 250%, or at least about 300%, relative to the control cell line, or
wherein there is an increase in the ratio of infectious viral particle production to total viral particle production of at least about 3%, such as at least about 20%, at least about 30%, at least about 40%, at least about 50%, or at least about 60%, relative to the control cell line.

3. The engineered cell line of claim 1, wherein the modification in the ISG15 gene results in deletion of the ISG15 gene from the engineered cell line or wherein the engineered cell line comprises decreased expression of the ISG15 gene as compared to the control cell line.

4. The engineered cell line of claim 1, wherein the engineered cell line is from a monkey cell, such as a Vero cell, or a mouse cell.

5. The engineered cell line of claim 3, wherein the ISG15 gene is deleted from the engineered cell line using a Clustered Regularly Interspaced Short Palindromic Repeats (CRISPR)-associated (Cas) system.

6. The engineered cell line of claim 1, wherein the virus is selected from influenza virus, such as an influenza A virus or an influenza B virus; dengue virus; yellow fever virus; respiratory syncytial virus (RSV); herpes simplex virus; human immunodeficiency virus (HIV); hepatitis virus; coronavirus; or a virus from the Rhabdoviridae family, such as rabies virus or vesicular stomatitis virus (VSV).

7. A method of increasing viral particle production comprising:
infecting an engineered cell line with a virus, wherein the engineered cell line comprises a modification in an ISG15 gene, wherein the modification in the ISG15 gene results in an increase in total viral particle production and/or infectious viral particle production as compared to a control cell line that is identical to the engineered cell line except for the modification in the ISG15 gene;
incubating the engineered cell line under conditions suitable for production of the virus by the engineered cell line; and
harvesting the virus produced by the engineered cell line.

8. The method of claim 7, wherein the engineered cell line increases viral particle production by at least about 20%, such as at least about 40%, at least about 50%, at least about 60%, or at least about 70%, at least about 80%, at least about 90%, at least about 100%, at least about 150%, at least about 200%, at least about 250%, or at least about 300%, relative to the control cell line.

9. The method of claim 7, wherein the modification in the ISG15 gene results in deletion of the ISG15 gene from the engineered cell line or wherein the engineered cell line comprises decreased expression of the ISG15 gene as compared to the control cell line.

10. The method according to claim 7, wherein the engineered cell line is a Vero cell line.

11. The method according to claim 7, wherein the virus is selected from influenza virus, dengue virus, yellow fever virus, RSV, herpes simplex virus, HIV, hepatitis virus, coronavirus, or a virus from the Rhabdoviridae family, such as rabies virus or VSV.

12. A method of identifying a gene for deletion in a cell or cell line, comprising:
infecting the cell or cell line with a virus;
detecting expression levels of multiple genes in the infected cell or cell line and comparing the expression levels to expression levels of the multiple genes in a control cell or cell line that is not infected with the virus;
identifying gene targets that are differentially expressed in the infected cell or cell line;
analyzing the differentially expressed gene targets to identify one or more gene targets involved in multiple protein-protein networks, wherein the multiple protein-protein networks comprise at least two of defense response, response to virus, viral genome replication, response to cytokine, response to type I interferon, regulation of viral genome replication, defense response to virus, cell death, viral life cycle, negative regulation of viral genome replication, and cellular response to cytokine stimulus; and
selecting at least one differentially expressed gene target for deletion in the cell or cell line,
wherein deletion of the at least one differentially expressed gene target increases viral particle production of the virus.

13. The method of claim 12, further comprising deleting the at least one differentially expressed gene target from the cell or cell line using a Clustered Regularly Interspaced Short Palindromic Repeats (CRISPR)-associated (Cas) system.

14. The method of claim 12, wherein the cell or cell line is a Vero cell, a Madin-Darby Canine (MDCK) cell, or a Human Embryonic Kidney (HEK) cell.

15. The method of claim 12, wherein the virus is selected from influenza virus, such as an influenza A virus or an influenza B virus; dengue virus; yellow fever virus; RSV; herpes simplex virus; HIV; hepatitis virus; coronavirus; or a virus from the Rhabdoviridae family, such as rabies virus or VSV.

16. The engineered cell line of claim 4, wherein the ISG15 gene is deleted from the engineered cell line using a Clustered Regularly Interspaced Short Palindromic Repeats (CRISPR)-associated (Cas) system.

17. The engineered cell line of claim 16, wherein the virus is selected from influenza virus, such as an influenza A virus or an influenza B virus; dengue virus; yellow fever virus; respiratory syncytial virus (RSV); herpes simplex virus; human immunodeficiency virus (HIV); hepatitis virus; coronavirus; or a virus from the Rhabdoviridae family, such as rabies virus or vesicular stomatitis virus (VSV).

18. The method of claim 8, wherein the modification in the ISG15 gene results in deletion of the ISG15 gene from the engineered cell line or wherein the engineered cell line comprises decreased expression of the ISG15 gene as compared to the control cell line.

19. The method according to claim 18, wherein the engineered cell line is a Vero cell line.

20. The method according to claim 19, wherein the virus is selected from influenza virus, dengue virus, yellow fever virus, RSV, herpes simplex virus, HIV, hepatitis virus, coronavirus, or a virus from the Rhabdoviridae family, such as rabies virus or VSV.


SEQUENCE LISTING (38 sequences)

SEQ ID NO: 1 - protein, 267 aa, Chlorocebus sabaeus
MKATVLTLAMLFLTGSQARHFWQEDEPPQTPWDRVKDLVTVYVEALKDSGKDYVSQFEGSALGKQLNLKLLDNWDSVTSTVSKLREQLGPVTQEFWDNLEKETEGLRQEMSKDLEEVKAKVQPYLDDFQKKWQEEMELYRQKVEPLRAELHDGARQKLHELHEKLSPLGEEVRDRARAHVDALRTHLAPYSDELRQRLAARLEALKENGGARLAEYHAKASEHLSTLSEKAKPALEDLRQGLLPVLESFKVSFLSALEEYSKKLSTQ

SEQ ID NO: 2 - protein, 99 aa, Chlorocebus sabaeus
MKVSAALLCLLLIAATFSPQGLAQPDAINAPVTCCYNFTNRKISVQRLASYRRITSSKCPKEAVIFKTIVAKEICADPKQKWVQDSMDHLDKQIQTPKP

SEQ ID NO: 3 - protein, 91 aa, Chlorocebus sabaeus
MKVSVAALAVILVATALCAPASASPYASDTTPCCFAYIARPLPRAHIKEYFYTSGKCSNPAVVFVTRKNRQVCANPEKKWVREYINSLEMS

SEQ ID NO: 4 - protein, 503 aa, Chlorocebus sabaeus
MVLEMLNPMHYNITSMVPEAMPAATMPILLLTGLFLLVWNYEGTSSIPGPGYCMGIGPLISHGRFLWMGIGSACNYYNRVYGEFMRVWISGEETLIISKSSSMFHIMKHNHYSSRFGSKLGLQCIGMHEKGIIFNNNPDLWKTTRPFFMKALSGPGLVRMVTVCAESLKTHLDRLEEVTNESGYVDVLTLLRRVMLDTSNMLFLRIPLDESAIVVKIQGYFDAWQALLIKPDIFFKISWLYKKYEKSVKDLKDAIEVLIAEKRRRISTEEKLEECMDFATELILAEKRGDLTRENVNQCILEMLIAAPDTMSVSLFFMLFLIAKHPNVEEAIMKEIQTVVGERDVKIDDMQKLKVMENFIYESMRYQPVVDLVMRKALEDDVIDGYSVKKGTNIILNIGRMHRLEFFPKPNEFTLENFAKNVPYRYFQPFGFGPRGCAGKYIAMVMMKAILVTLLRRFHVKTLQGQCVERIQKIHDLSSHPDETKNMLEMIFTPRNSDRCLEH

SEQ ID NO: 5 - protein, 101 aa, Chlorocebus sabaeus
MTSKLAVALLAAFLLSAALCEGAVLPRSAKELRCQCIKTYSKPIHPKFIKELRVIESGPHCVNTEIIVKLSDGRELCLDPKVPWVSRVVEKFLKRAESQNS

SEQ ID NO: 6 - protein, 353 aa, Chlorocebus sabaeus
MAATCEISNIFSNYVSAMYSSEDSTLAPVPPAAAFGADDLVLTLSNPQMSLEGTEKTSWSGEQPQFWSKTQVLDWISYQVEKNKYDASAIDFSRCDMDGATLCNCALEELRLVFGPLGDQLHAQLRDLTSSSSDELSWIIELLEKDGMAFQEALDPGPFDQGSPFAQELLDDSQQASPYHPGSCGAGAPSPGSSDVSTAGTGASRSSHSSDSGGSDVDLDPTDGKLFPRDGFPDCKKGDPKHGKRKRGRPRKLSKEYWDCLEGKKSKHELNEGLMKWENRHEGVFKFLRSEAVAQLWGQKKKNSNMTYEKLSRAMRYYYKREILERVDGRRLVYKFGKNSSGWKEEEVLQSRN

SEQ ID NO: 7 - protein, 380 aa, Chlorocebus sabaeus
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPTPSAGAYSRAGIVKTMTGSRAQSTGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLSGGLPEAATPESEEAFTLPLLNDPEPKPSVEPVKSISSMELKAEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 8 - protein, 917 aa, Chlorocebus sabaeus
MLCWGYWSLGQPGISTNLQGIVAEPQVCGFISDRSVKEVACGGNHSVFLLEDGEVYTCGLNTKGQLGHEREGNKPEQIGALADQHIIHVACGESHSLALSDRGQLFSWGAGSDGQLGLMTTEDSVAVPRLIQKLNQQTILQVSCGNWHCLALAADGQFFTWGKNSHGQLGLGKEFPSQASPQRVRSLEGIPLAQVAAGGAHSFALSLSGAVFGWGMNNAGQLGLSDEKDRESPCHVKLLRTQKVVYISCGEEHTAVLTKSGGVFTFGAGSCGQLGHDSMNDEVNPRRVLELMGSEVTQIACGRQHTLAFVPSSGLIYAFGCGARGQLGTGHTCNVKCPSPVKGYWAAHSGQLSARADRFKYHIVKQIFSGGDQTFVLCSKYENSSPAVDFRTMNQAHYTSLINDETIAVWRQKLSEHNNANTINGVVQILSSAACWNGSFLEKKIDEHFKTSPKIPGIDLNSTRVLFEKLMNSQHSMILEQILNSFESCLIPQLSSSPPDVEAMRIYLILPEFPLLQDSKYYITLTIPLAMAILRLDTNPSKVLDNWWSQVCPKYFMKLVNLYKGAVLYLLRGRKTFLIPVLFNNYITAALKLLEKLYKVNLKVKHVEYDTFYIPEISSLVDIQEDYLMWFLHQAGMKARPSIIQDTVTLCSYPFIFDAQAKTKMLQTDAELQMQVAVNGANLQNVFMLLTLEPLLARSPFLVLHVRRNNLVGDALRELSIHSDIDLKKPLKVIFDGEEAVDAGGVTKEFFLLLLKELLNPIYGMFTYYQDSNLLWFSDTCFVEHNWFHLIGITCGLAIYNSTVVDLHFPLALYKKLLNVKPGLEDLKELSPTEGRSLQELLDYPGEDVEETFCLNFTICRESYGVIEQKKLIPGGDNVTVCKDNRFTVYISTCERLNIPTDSGQAIYKNGAETHSL

SEQ ID NO: 9 - protein, 1023 aa, Chlorocebus sabaeus
MERRSRRKSRRNGRSTAGQTAASQPAKSPDAQLWLFPSAAGFYRALLRRAEVTRQICCSPRRLAVLERGGAGVQVHQVLAGSGGARTPKCIKLGKNMKIHSMDQGADHMLILSSDGKPFEYDYSMKHLRSESILQEKKIIQITCGDYHSLALSKGGELFAWGQNLHGQLGVGRKFPSTTTPQIVEHLAGVPLAQISAGEAHSMALSMSGNIYSWGKNEFGQLGLGHTESKDSPSLIEALDNQKVEFLACGGSHSALLTQDGLLFTFGAGKHGQLGHNSTQNELRPCLVAELVGNRVTQIACGRWHTLAYVSDLGKVFSFGSGKDGQLGNGGTLDQLIPLPVKVSSSEELKLESHTSEKELIMIAGGNQSILLWIKKENSYVNLKRTIPTLNEGTVKRWIADVETKRWHSTKREIQEIFSSPACLTGSFLRKRRTTEMMPVYLDLNKARNIFKELTQKDRITNMITTCLKDNLLKRLPFHSPHQEALEVFFLLPECPVMHLSNNWESLVVPFAKVVCKMSDPSSLVLEEYWATLQESTFSKLVQMFKTAVVCQLDYWDESAEENGNVQALLEMLKKLHRVNQMKCQLPESIFQVDELLYRLNFFVEVCRRCLWKMTVDTSENAGCWVIFSHFPFIFNHLSKIKLLHTDTLLKIEGKKHKAYLMSAAIEEERESEFALMPTFGLTVRRNHLIEDVLNQLSQFENEDLRKELWVSFSGEIGYDLVGVKREFFYCLFEEMIQPEYGMFMYPEGASCMWFPVRPKFEKKRYFFFGLLCGLSLFNCNVANLPFPLALFKKLLDQMPSLEDLKELSPDLGKNLQTLLDDEGDNFEEVFYIHFNVHWDRNDINLIPNGSSIIVNQTNKRDYVSKYIDYIFNDSVKAVYEEFRRGFYKMCDEDIIKLFHPEELKDVIVGHTDYDWKTFEKNARYEPGYNSSHPTIVMFWKAFHKLTLEEKKKFLVFLTGTDRLQTKDLKNMKITFCCPESWNERDPMRALTCFSVLFLPKYSTMEAVEEALQVAINNNRGFG

SEQ ID NO: 10 - protein, 478 aa, Chlorocebus sabaeus
MSTNGDNHQVKDSLEQLRCHFTWELFIEDDEMPDLENRVLDQIEFLDTKYNVGIHNLLAYVKHLKGQNEEALKSLKEAEDLMQKEHANQASVRSLVTWSNFAWVYYHMGRLAEAQAYLDKVENICKKPSNPFRYRMECPEIDCEEGWALLKCGGKNYERAKACFEKALEGDHENPEFSTGYAISAYRLDGFKLATKGYRQFSLLPLRQAVSLNPDNGYLKVLLALKLQDNGQEAEGEKYLEEALANMSSQTYVFRYAAKFYRRKGAVDKALELLKKALQETPTSVLLHHQIGLCYKTQMIQLKEATKGQPRGQNREKIDKMIRLAIFHFESAVEKKPTFEVAHLDLARMYIEAGNHRKAEETFQKLLCMKPVVEETMQDIHLQYARFQEFQKKSEINAIIHYLKAIKIEQASFIRDKSINSLKKLVLKKLQRNALDLESLSLLGFVYKLKGNMNEALEYYERALRLAADFENSVRQGP

SEQ ID NO: 11 - protein, 472 aa, Chlorocebus sabaeus
MSENTKNSLESSLRQLKCHFTWNLIEGENSLDDFEDKVFYRTEFQNREFKATMCNLLAYLKHLKGQNEEALECLCKAEELIQQEHADQAEIRSLVTWGNYAWVYYHMGRLSDAQIYVDKVKHICEKFSSPYRIESPELDCEEGWTRLKCGGNQNERAKVCFEKALEKKPKNPEFTSGLAIASYRLDNWPPSQNAIDPLRQAIQLNPDNQYLKVLLALKLHKMREEGEEEGEGEKLVQEALEKAPGITDVLRSAAKFYRRKDEPDKAIELLKKALEYLPNNAYLHCQIGCCYRAKVLQVMNLRQNGIYGKRKLLELIGHAVAHLKKADEANDNLFRVCSILASLHALADQYEEAEYYFQKEFSKELTPVAKQLLHLRYGNFQLYQMKCEDKAIHHFIEGVKINQKSREKEKMKDKLQKIAKMRLSKNGADSEALHVLAFLQELNKKMQQADEDSERGLESGSLIPSASSWNGE

SEQ ID NO: 12 - protein, 435 aa, Chlorocebus sabaeus
MYNLLAYIKHLDGKNEAALECLRQAEELIQQEHADQAEIRSLVTWGNYAWVYYHLGRLSDAQIYVDKVKQTCKKFSNPYSIEYPELDCEEGWTQLKCGRNERAKVCFEKALEEKPNNPEFSSGLAIAMYHLDNNPEKQFSTDVLKQAIELSPDNQYVKVLLGLKLQKMNKEAEGEQLVEEALEKAPCQTDVLRSAAKFYRRKGDLDKAIELFQRALESTPNNGYLYHQIGCCYKAKVRQMQNTGESEASGNKEKIEALKQYAMDYSNKALEKGLNPLDAYSDLAEFLEAECYQTPFSKEDPDAEKQQSHQHYYNLQKYNGKSEDTALQRGLEGLSISKKSTEKEEIKDQPQNVSENLLPQNAPNYWYHQGLIHKQNGDLLQAVKCYEKELGRLLRNAPSGIGSFFLSASELEDGSEEMGQDAVSSNPRELVSNSE

SEQ ID NO: 13 - protein, 504 aa, Chlorocebus sabaeus
MALAPERAAPRVLFGEWLLGEISSGCYEGLQWLDEARTCFRVPWKHFARKDLSEADARIFKAWAVARGRWPPSSRGGDPPPPEAEAAERASWKTNFRCALRSTRRFVMLRDNSGDPADPHKVYALSPELGWREGPGTDQTEAEAPAAVRAPQGRPPGPFLAHRDAGLQAPGPLPAPAGDKGDLLLQAVQQSCLADHLLTASWGADPVPAQAPGEGQEGLPLTGACAGGPGLPAGELCTWAVEATPSPGSQPAALMTGEATAPEPLHQVEPYLAPSPSACTAVQEPSPGALDVTIMYKGRTVLQKVVGHPSCMFLYGPPDPAVRATDPQQVAFPSPAELPDQKQLRYTEELLRHVAPGLQLELRGPQLWARRMGKCKVYWEVGGPPGSASPSTPACLLPRNCDTPIFDFRVFFRELVEFRARQRRGSPCYTIYLGFGQDLSARRPKEKSLVLVKLEPWLCRVHLEGTQREGVSSLDSSSLSLCLSSTNSLYDDIECLLMELEQPV

SEQ ID NO: 14 - protein, 165 aa, Chlorocebus sabaeus
MSWDLKVKMLGGNEFQVSLSSSMSVSELKAKIAQKIGVHAFQQRLAVHPSGATLQDRVPLANQGLGPGSTVLLVVDRCDEPLSILVRNDKGRSSTYEVQLTQTVAHLKQQVSRQEGVQDDLFWLTFEGKPLENQLPLGEYGLKPLSTVFMNLRLRGGGTEPGGQS

SEQ ID NO: 15 - protein, 165 aa, Homo sapiens
MGWDLTVKMLAGNEFQVSLSSSMSVSELKAQITQKIGVHAFQQRLAVHPSGVALQDRVPLASQGLGPGSTVLLVVDKCDEPLSILVRNNKGRSSTYEVRLTQTVAHLKQQVSGLEGVQDDLFWLTFEGKPLEDQLPLGEYGLKPLSTVFMNLRLRGGGTEPGGRS

SEQ ID NO: 16 - protein, 161 aa, Mus musculus
MAWDLKVKMLGGNDFLVSVTNSMTVSELKKQIAQKIGVPAFQQRLAHQTAVLQDGLTLSSLGLGPSSTVMLVVQNCSEPLSILVRNERGHSNIYEVFLTQTVDTLKKKVSQREQVHEDQFWLSFEGRPMEDKELLGEYGLKPQCTVIKHLRLRGGGGDQCA

SEQ ID NO: 17 - protein, 174 aa, Canis lupus
MLEPTAMAGNLTVKMLGGEEFLVPLRDSMLASELKQQIALKTGVPAFQQRLATHPAGTVLQDGISLIRQGLCPGSTVLLVVKNCNDPLSILVRNNKGRSIAYEVWLTQTVAELKQQVCQQEHVQADLFWLTFEGKPMEDKHQLGEYGLTPQCTVFMNLRLRGGGGNWAGPGGQC

SEQ ID NO: 18 - protein, 456 aa, Chlorocebus sabaeus
MTTTFLQTSSSTFGGGSTRGGSLLAGGGGFGGGSLYGGGGSRTISASSARFVSSGSGGGYGAGMRVCGFGGGAGSAFGGGFGGGIGGGFGGGFGGGDGGLLSGNEKITMQNLNDRLASYLDKVRALEEANADLEVKIRDWYQKQAPTSPERDYSQYFKTTEELRDKIMATTIDNSRVILEIDNARLAADDFRLKYENELALRQSVEADINGLRRVLDELTLAKTDLEMQIEGLNEELAYLKKNHEEEMKEFGSQLAGQVNVEMDAAPGVDLTRVLAEMREQYEAMAEKNRRDAEAWFFSKTEELNKEVASNTEMIQTSKTEITDLRRTMQELEIELQSQLSMKAGLENSLAETECRYATQLQQIQGLIGGLEAQLSELRCEMEAQNQEYKMLLDIKTRLEQEIATYRSLLEGQDAKMADIGIREASSGGGGSSSNFRINVEESVDGKVVSSRKREI

SEQ ID NO: 19 - protein, 400 aa, Chlorocebus sabaeus
MTSYSYRQSSATSSFGGLGGGSVRFGPGVAFRAPSMHGGSGGRGVSVSSARFVSSSSSGGYGGGYGGVLAASDGLLAGNEKLTMQNLNDRLASYLDKVRALEAANGELEVKIRDWYQKQGPGPSRDYSHYYTTIQDLRDKILGATIENSRIVLQIDNARLAADDFRTKFETEQALRMSVEADINGLRRVLDELTLARTDLEMQIEGLKEELAYLKKNHEEEISVLRGQVGGQVSVEVDSAPGTDLAKILSDMRSQYEVMAEQNRKDAEAWFTSRTEELNREVAGHTEQLQISRSEVTDLRRTLQGLEIELQSQLSMKAALEDTLAETEARFGAQLAHIQALISGVEAQLGDVRADSERQNQEYQRLMDIKSRLEQEIATYRSLLEGQEDHYNNLSASKVL

SEQ ID NO: 20 - protein, 661 aa, Chlorocebus sabaeus
MVLSEVDIVKADPAAASQPLLLNGDADVAQKSPGSVAENNLCSQYEEKVRPCIDLIDSLRALGVEQDLALPAIAVIGDQSSGKSSVLEALSGVALPRGSGIVTRCPLVLKLKKLVNEDEWRGKVSYQDYEIEILDASEVEKEINKAQNTIAGEGMGISHELITLEISSRDVPDLTLIDLPGITRVAVGNQPPDIGYKIKTLIRKYIQRQETINLVVVPSNVDIATTEALSMAQEVDPEGDRTIGILTKPDLVDKGTEDKVVDVVRNLVFHLKKGYMIVKCRGQQEIQDQLSLSEALQREKIFFEDHPHFRDLLEEGKATIPCLAEKLTSELIAHICKSLPLLENQIKESHQGITEELQKYGVDIPEDENEKMFFLIDKINTFNQDITALIQGEETVGEDDSRLFTRLRREFHKWGIIIENNLQEGHKITSRKMQKFENQYRGRELPGFVNYRTFETIIKQQIKALEEPAVNMLHTVTDMVRLAFADVSMKNFEELFNLHRTAKSKIEDIRTEQEREGEKLIRLHFQMEQIVYCQDQVYRGALQKVREKELEEEKKKKSWDVGTFQPSSTDSSMEEIFQHLMAYHQEASKRISSHIPLIIQFFMLQTYGQQLQKAMLQLLQDKDTYSWLLKERSDTSDKRKFLKERLARLTQARRRLAQFPG

SEQ ID NO: 21 - protein, 427 aa, Chlorocebus sabaeus
MRAGATGRAMDGPRLLLLLLLGVSLGGAKEACPTDLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVADVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 22 - protein, 604 aa, Chlorocebus sabaeus
MLARALLLCAVLALGHAANPCCSYPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNVDYGYKSWEAFSNLSYYTRALPPVPDDCPTPLGVKGKKQLPDSNEIVEKFLLRRKFIPDPQGTNMMFAFFAQHFTHQFFKTDHKRGPAFTKGLGHGVDLNHIYGETLDRQHKLRLFKDGKMKYQIIDGEMYPPTVKDTQAEMIYPPQVPEHLQFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDNFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSRLDDINPTVLLKERSTEL

SEQ ID NO: 23 - protein, 600 aa, Chlorocebus sabaeus
MPSPRMVRWFHRDLSGLDAETLLKGRGVHGSFLARPSRKNQGDFSLSVRVGDQVTHIRIQNSGDFYDLYGGEKFATLAELVEYYTQQQGVLQDRDGTVIHLKYPLNCSDPTSERWYHGHMSGGQAETLLQAKGEPWTFLVRESLSQPGDFVLSVLSDQPKAGPGSPLRVTHIKVMCEGGRYTVGGSETFDSLTDLVEHFKKTGIEEASGAFVYLRQPYYATRVNAADIENRVLELNKKKESEDTAKAGFWEEFESLQKQEVKNLHQRLEGQRPENKGKNRYKNILPFDHSRVILQGRDSNIPGSDYINANYIKNQLLGPDENTKTYIASQGCLEATVNDFWQMAWQENTRVIVMTTREVEKGRNKCVPYWPEVGTQRVYGPYSVTNCGEHDTTEYKLRTLHVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHSKNKREEKVKKQRSADKEKSKGSLKRK

SEQ ID NO: 24 - protein, 1115 aa, Chlorocebus sabaeus
MAKATSGAAGLRLLLLLLLLPLLGKVALGLYFSRDAYWEKLYVDQPAGTPLLYVHALRDAPEEVPSFRLGQHLYGTYRTRLHENNWICIQEDTGLLYLNRSLDRSSWEKLSGRNRGFPLLTVYLKVFLSPTFLREGECQWPGCARVYFSFFNTSFPACTSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWTQQTFRVEHWPNETSVQANGSFVRATVHDYRLVLNRNLSISENRTMQLAVLVNDSDFQGPGAGVLLLHFNVSVLPVSLHLPSSYSLSVSRRARRFAQIGKVCVENCQAFSGINVQYELHSSGANCSTLGVVTSAEDTSGILFVNDTKALRRPKCAELHYMVVATDHQTSRQAQAQLLVTVEGSYVAEEAGCPLSCAVSKRRPECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHRYHKFAHKPPIPSAEMTFRRPAQAFPVSYSSSGTRRPSLDSMENQVSVDAFKIPEDPKWEFPRKNLVLGKTLGEGEFGKVVKATAFRLKGRAGYTTVAVKMLKENASPSELRDLLSEFNLLKQVNHPHVIKLYGACSQDGPLLLIVEYAKYGSLRGFLRESRKVGPGYLGSGGSRNSSSLDHPDERALTMGDLISFAWQISRGMQYLAEMKLVHRDLAARNILVAEGRKMKISDFGLSRDVYEEDSYVKRSKGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKSRDYLDLAASTPSDSLLYDDGLSEEETPLVDCNNAPLPRALPSTWIENKLYGMSDPNWPGESPVPLTRADGTNTGFPRYANDSVYANWMLSPSAAKLMDTFDS

SEQ ID NO: 25 - protein, 2340 aa, Chlorocebus sabaeus
MKNIYCLILKLVNFATLGCLWISVVQCTVLNSCLKSCVTNLGQQLDLGTPHNLSEPCIQGCHFWNSVDQKNCALKCNDTYATICERESCEVGCSSAEGAYEEEVLGNADLPTAPFASSIGSHDMTLRWKSANFSGVKYIIQWKYAQLLGSWTYTKTVSKLSYVVEPLHPFTEYIFRVVWIFTAQLQLYSPPSPSYRTHPHGVPETAPLIRNIESSSPDTVEVSWDPPQFPGGPILGYNLRLISKNQKLDAGTQRTSFQFYSTLPNTMYRFSTAAVNEVGEGPEAESSITTSSSAVQEEEQWLFLSRKTSLRKRSLKHLVDEAHCLRLDAIYHNITGISVDVHQQTVYFSEGTLIWVKKAVNMSDVSDLKIFYRGSGLISSISVDWLYQRMYFIMEELVCVCDLENCSNIEEITPPSISAPRKIVADSYNGYVFYLLRDGIYRADLPVPSGRRAETVRIVESCTLKDFAIKPQSKRIVYFNDTAQVFMSTFLDGSASHLILPRIPFADVKSFACENNDFLVTDGKVIFQQDALSFNEFIVGCDLSHIEEFGFGNLVIFGSSSQLHPLPGRPQELSVLFGSHQAFVQWKPPALAIGASPSAWQNWTYEVKVSTQDPPEVTRIFSNISGTMLNVPELQSATKYKVSVRASSPKRPGPWTEPSVGTTLVPASEPPFIMAVKEDGLWSKPLNSFGPGEFLSSDIGNVSDMDWYNNSLYYSDTKGDVFVWLLNGMDISENYHLPGIAGAGALAFEWLGHFLYWAGKTYVIQRQSVLTGHTDIVTHVKLLVNDMVVDSVGGYLYWTTLYSVESTRLNGESSLILQAQPWFSGKKVIALTLDLSDGLLYWLVQDSQCIHLYTAVLRGQSTGDTTITEFAAWSTSEISQNALMYYSGRLFWINGFRIITTQEIGQRTSVSVLEPAKFNQFTIIQTSLKPLPGNFSFTPKVIPDSVQESSFRIEGNASSFQILWNGPPAVDWGVVFYSVEFSAHSKFLASERHSLPVFTVEGLEPYALFNLSVTPYTYWGKGPKTALSLRAPETVPSAPENPRIFILPSGKCCNKNEVVVEFRWNKPKHENGVLTKFEIFYKISNQSITNKTFEDWIAVDVTPSVMSFQLEGMSPRCFVAFQVRAFTSKGPGSFADVVKSTTSEINPFPHLITLLGNEIAFLDMDQNQVVWTFSAERVISAICYTADNELGYYAEGDSLFLLNLHNRSSSELFRDSLVFDITVITIDWISRHLYFALKESQNGMQIFDVDLEHKVKHPREVKIHNRNSTIISFSVYPLLSRLYWTEVSSFGYQMFYYSIINCTLHRILQPTATNQQNKRNQCSCNVTEFELSGAMAIDTSNLEKSLIYFAKTQEIWAMDLEGCHCWRVITVPAMLGKTLASLTVDGDFIYWIIIAKDSTRIYQAKKGNGAIVSQVKALRSRHILAYSSVMQPFPGKAFLSLASETVEPTILNATNTSLTIRLPLAKTTLTWYGITSPTPTYLVCYAEVNDRKNSSDLKYRILEFQDSIALIEDLQPFSTYMIQIAVKNYYSDPLEHLPPGKEIWGKTKNGVPEAVQLINTTVRSDTSLIISWKESHKPNGPQESVRYQLAISHLAPIPETPLRQSEFPNGRLTLLVTGLSGGNIYVLKVLACHSEEMWCTESHPVTVEMFNTPEKPYSLVPENTSLQFNWKAPLNVNLIRFWAELQKWKYNEFYHVKTSCNQGPAYVCNITNLQPHTSYNVRVVVVYKTGENSTSLPESFKTKAGVPSKPGIPKLLEGSKNSIQWEKAEDNGCRIIYYILEIRKSTSNLQNQNLRWKMTFNGSCSSICTWKSKNLKGIFQFRVVAANNLGFGEYSGISENTVLVGDDFWIPETSFILSIIVGIFLVITIPLTFVWLRRLKNQKSAKEGLTVLINEDKELAELRGLAAGVGLANACYAIHTLPTQEEIENLPAFPREKLTLRLLLGSGAFGEVYEGTAVDILGAGSGEIKVAVKTLKKGSTDQEKIEFLKEAHLMSKFNHPNILKQLGVCLLNEPQYIILELMEGGDLLTYLRKARMATFYGPSLTLVDLVDLCVDISKGCVYLEQMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYQCRDEANTSGVINENFEGEDGDVICLNSDDIMPVALMETKNREGLNYMVLATECGQGEEKSEGPLGSQESKSCGLRKEEKEPHADKDFCQEKQVAYCPPGKPEGLNYACLSHTGYGDGAD

SEQ ID NO: 26 - protein, 314 aa, Chlorocebus sabaeus
MGSGRSAGGCRGAALEVLLALGAALLAVGLASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK

SEQ ID NO: 27 - protein, 319 aa, Chlorocebus sabaeus
MYNMMETELKPPGPQQTSGGGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 28 - protein, 314 aa, Chlorocebus sabaeus
MRIAVISFCLLGIAYALPVKQADSGSSEEKQLYNKYPDAVATWLKPDPSQKQNLLAPQNAVSSEETNDFKQETLPSKSNESHDHMDDVDDEDDDDHVDSQDSIDSNDSDEVDDTDDSHQSDESHQSDESDELVTDFPTDLPATEVFTPVVPTVDIYDGRGDSVAYGLRSKSKKFRRPDIQYPDATDEDITSHVESEELNGAYKAIPVAQGLNVPSDWDSRGKDSHETSQLDDHSAETHSHKHSRLYKRKASDDSNEHSDVIDSQERSKISREFHSHEFHSHEDMLVVDPKSKEEDKHLKFRISHELDSASSEVN

SEQ ID NO: 29 - protein, 233 aa, Chlorocebus sabaeus
MSTESMIRDVELAEEALPRKTAGPQGSRRCWFLSLFSFLLVAGATTLFCLLHFGVIGPQREEFPKDPSLFSPLAQAVRSSSRTPSDKPVAHVVANPQAEGQLQWLNRRANALLANGVELTDNQLVVPSEGLYLIYSQVLFKGQGCPSNHVLLTHTISRIAVSYQTKVNLLSAIKSPCQRETPEGAEAKPWYEPIYLGGVFQLEKGDRLSAEINLPDYLDFAESGQVYFGIIAL

SEQ ID NO: 30 - protein, 277 aa, Chlorocebus sabaeus
MCVGARRLGRGPCAALLLLGLGLSTTAKLHCVGDTYPSNDRCCQECRPGNGMVSRCNRSQNTVCRPCGPGFYNDVVSAKPCKACTWCNLRSGSERKQPCTATQDTVCRCRAGTQPLDSYKPGVDCAPCPPGHFSPGDNQACKPWTNCTLAGKHTLQPASNSSDAICEDRDPTPTQPQETQGPPARPTTVQPTEAWPRTSQRPSTRPVGVPRGPAVAAILGLGLALGLLGPLAMLLALLLLRRDQRLPPDAPKAPGGGSFRTPIQEEQADAHSTLAKI

SEQ ID NO: 31 - protein, 294 aa, Chlorocebus sabaeus
MKQWKTRLGSGLESGPMALEQNLSDLQLQAAVEVAGDLEVDCYRAPCSESQEELALQHFMKEKLLAELEGKLRVFENIVAVLNKEVEASHLALATSIHQSQVDRERILSLEQRVVELQQTLAQKDQALGKLEQSLRLMEEASFDGTFLWKITNVTRRCHESACGRTVSLFSPAFYTAKYGYKLCLRLYLNGDGTGKRTHLSLFIVIMRGEYDALLPWPFRNKVTFMLLDQNNREHAIDAFRPDLSSASFQRPQSETNVASGCPLFFPLNKLQSPKHAYVKDDTMFLKCIVETST

SEQ ID NO: 32 - protein, 847 aa, Chlorocebus sabaeus
MEPWKQCAQWLIHCKVLPANHRVTWDSAQVFDLAQTLRDGVLLCQLLNNLRAHSINLKEINLRPQMSQFLCLKNIRTFLTACCETFGMRKSELFEAFDLFDVRDFGKVIETLSRLSRTPIALATGIRPFPTEESINDEDVYKGLPDLIDETLVEDEEDLYDCVYGEDEGGEVYEDLMKAEEAHQPKCPENDIRSCCLAEIKQTEEKYTETLESIEKYFMAPLKRFLTAAEFDSVFMNIPELVKLHRNLMQEIHDSIVNKNDHNLYQVFINYKERLVIYGQYCSGVESAISSLDYISKTKEDVKLKLEECSKRANNGKFTLRDLLVVPMQRVLKYHLLLQELVKHTTDATEKANLKLALDAMKDLAQYVNEVKRDNETLREIKQFQLSIENLNQPVLLFGRPQGDGEIRITTLDKHTKQERHIFLFDLAVIVCKRKGDNYEMKEIIDLQQYKIANNPTTDKENKKWSYGFYLIHTQGQNGLEFYCKTKDLKKKWLEQFEMALSNIRPDYADSNFHDFKMHTFTRVTSCKVCQMLLRGTFYQGYLCFKCGARAHKECLGRVDNCGRVNSGEQGTLKLPEKRTNGLRRTPKQVDPGLPKMQVIRNYSGTPPPALHEGPPLHLQAGDTVELLRGDAHSLFWQGRNLASGEVGFFPSDAVKPCPCVPKPVDYSCQPWYAGAMERLQAETELINRVNSTYLVRHRTKESGEYAISIKYNNEAKHIKILTRDGFFHIAENRKFKSLMELVEYYKHHSLKEGFRTLDTTLQFPYKEPEHSTGQRVNRAGNSLLSPKVLGIAIARYDFCARDMRELSLLKGDVVKIYTKMSANGWWRGEVNGRVGWFPSTYVEEDE

SEQ ID NO: 33 - DNA, 24 bp, synthetic construct
  source 1..24: mol_type = other DNA
accagcattcgagcaagatcaagg

SEQ ID NO: 34 - DNA, 23 bp, synthetic construct
  source 1..23: mol_type = other DNA
ggaaaccgaaacttggccaccgg

SEQ ID NO: 35 - DNA, 21 bp, synthetic construct
  source 1..21: mol_type = other DNA
gtcccagctctgcagacatta

SEQ ID NO: 36 - DNA, 20 bp, synthetic construct
  source 1..20: mol_type = other DNA
gagctcggccaggttctaag

SEQ ID NO: 37 - DNA, 20 bp, synthetic construct
  source 1..20: mol_type = other DNA
cctcgaggctgtaactgcaa

SEQ ID NO: 38 - DNA, 20 bp, synthetic construct
  source 1..20: mol_type = other DNA
accataggggtgttttccgt